(19) United States
(12) Patent Application Publication
(10) Pub. No.: US 2015/0133388 A1
(43) Pub. Date: May 14, 2015

(54) ACETYLATED CRYSTALLIN POLYPEPTIDES AND MIMETICS THEREOF AS THERAPEUTIC AGENTS

(71) Applicant: Case Western Reserve University, Cleveland, OH (US)

(72) Inventors: Ram Nagaraj, Cleveland, OH (US); Rooban B. Nahomi, Cleveland, OH (US)

(21) Appl. No.: 14/600,564

(22) Filed: Jan. 20, 2015

Related U.S. Application Data
(63) Continuation-in-part of application No. PCT/US2013/051504, filed on Jul. 22, 2013.
(60) Provisional application No. 61/674,010, filed on Jul. 20, 2012.
(60) Provisional application No. 61/773,623, filed on Mar. 6, 2013.

(51) Int. Cl. A61K 38/17

(52) U.S. Cl. CPC A61K 38/1703
USPC 514/17.8; 514/18.9; 514/1.1; 514/20.8; 514/17.7

(57) ABSTRACT

A method of inhibiting, reducing, and/or treating pathological apoptosis and/or protein aggregation in a subject includes administering to the subject an amount of a therapeutic polypeptide effective to inhibit, reduce, and/or treat the pathological apoptosis and/or protein aggregation. The therapeutic polypeptide including at least one of acetylated αA-crystallin, acetylated αB-crystallin, acetylated fragments thereof having molecular chaperone activity, and/or polypeptide mimetics thereof that can inhibit pathological protein aggregation and/or pathological apoptosis.

### RELATED APPLICATION

This application is a Continuation-in-Part of PCT/US2013/051504, filed Jul. 22, 2013, which claims priority from U.S. Provisional Application Nos. 61/674,010, filed Jul. 20, 2012 and 61/773,623, filed Mar. 6, 2013, the subject matter of which are incorporated herein by reference in their entirety.

### GOVERNMENT FUNDING

This invention was made with government support under Grant No. EY 016219 awarded by The National Institutes of Health. The United States government has certain rights to this invention.

### BACKGROUND

Crystallins are water-soluble proteins that are highly refractive and are related to metabolic enzymes and stress-protective proteins. α, β, and γ crystallins are the major protein components of the vertebrate eye lens with alpha crystallin being both a molecular chaperone as well as a structural protein, whilst beta and gamma crystallins are structural proteins. Lenticular proteins, such as the abundant water-soluble crystallins cannot be replaced and thus must last the lifetime of the organism. βB2 crystallin has been demonstrated as being essential for maintaining the high solubility of crystallins in the eye lens. Its expression does not appear to be induced in other tissues upon changes in physiological condition that occur during wounding.

There are two α crystallin genes, αA and αB (for acidic and basic, respectively), encoding proteins that share approximately 60% sequence identity. αA and αB crystallins have two domains, α crystallin domain and an alphα-crystallin-HSP domain. Two other domains share homology to the α-crystallin-HSP domain, namely the HSP20 domain and IbpA domain. Alpha crystallins can be induced by heat shock and are members of the small heat shock protein (sHSP) family. They act as molecular chaperones and hold unfolded or misfolded proteins in large, water soluble low molecular weight aggregates. These heterogeneous aggregates consist of 30-40 subunits of α crystallins in which the αA and αB subunits are present in a 3:1 ratio.

Alpha crystallins are present in all animal kingdoms but not in all organisms. Only αB crystallin has been found to be stress inducible. The expression of αA crystallin is essentially limited to the eye lens with only traces found in some other tissues. As such, αA crystallin is an essentially eye lens specific member of the family. αB crystallin is more widely expressed and is particularly abundant in brain, heart and muscle.

β crystallins are members of the beta/gamma-crystallin family. There are at least 5 different proteins comprising the β crystallins. The beta/gamma-crystallin family of proteins contains a two-domain β-structure, folded into four very similar “Greek Key” motifs. ƒ3 crystallins form homo/heterodimer, or complexes of higher order. The structure of β-crystallin oligomers appears to be stabilized through interactions between their N-terminal arms. βB2 crystallin contains a duplication of the XTALbg domain. At least 5 gamma crystallins have been identified in bovine and rat lens.

### SUMMARY

Embodiments described herein relate to compositions and methods of inhibiting, reducing, and/or treating pathological apoptosis and/or pathological protein aggregation and to methods and compositions of treating diseases, disorders, and/or conditions associated with pathological apoptosis and/or pathological protein aggregation. The compositions include therapeutic polypeptides (or proteins) that can inhibit pathological protein aggregation and/or pathological apoptosis in cells, such as neural cells, ganglion cells, and epithelial cells. The therapeutic polypeptides can include acetylated αA-crystallin, acetylated αB-crystallin, acetylated fragments thereof having molecular chaperone activity, and/or polypeptide mimetics thereof that can inhibit pathological protein aggregation and/or pathological apoptosis.

In some embodiments, the therapeutic polypeptide can have an amino acid sequence with a sequence identity of SEQ ID NO: 9, SEQ ID NO: 10, SEQ ID NO: 11 or SEQ ID NO: 12.

In other embodiments, the therapeutic polypeptide can have an amino acid sequence with a sequence identity selected from the group consisting of SEQ ID NO: 1, SEQ ID NO: 2, SEQ ID NO: 3 and SEQ ID NO: 4.

In still other embodiments, the therapeutic polypeptide can have an amino acid sequence with a sequence identity selected from the group consisting of SEQ ID NO: 5, SEQ ID NO: 6, SEQ ID NO: 7, and SEQ ID NO: 8.

In yet other embodiments, the therapeutic polypeptide can have an amino acid sequence with a sequence identity selected from the group consisting of SEQ ID NO: 3, SEQ ID NO: 4, SEQ ID NO: 7, and SEQ ID NO: 8.

In some embodiments, the pathological apoptosis and/or pathological protein aggregation is associated with or results from an optical neuropathy, glaucoma, or cataracts, an inflammatory condition, and/or a brain injury.

Other embodiments described herein relate to an ophthalmic composition or preparation that includes a therapeutically effective amount of a therapeutic polypeptide effective to inhibit protein aggregation and/or epithelial cell apoptosis in the subject's eye. The therapeutic polypeptide includes at least one of acetylated αA-crystallin, acetylated αB-crystallin, acetylated fragments thereof having molecular chaperone activity, and/or polypeptide mimetics thereof. The therapeutic polypeptide can have an amino acid sequence with a sequence identity of SEQ ID NO: 9, SEQ ID NO: 10, SEQ ID NO: 11 or SEQ ID NO: 12.

### BRIEF DESCRIPTION OF THE DRAWINGS

FIGS. 1(A-C) illustrate lysine acetylation differentially alters chaperone function in α-crystallin peptides. Native and acetyl αA- and αB-crystallin peptides were assessed for their chaperone function using four client proteins: citrate synthase (CS), insulin (IN), β-crystallin (BLC), and γ-crystallin (GC). The ratios of the client proteins to the peptides are given in the Experimental Procedures except for the scrambled peptide, which was tested at a two times higher concentration. FIG. 1A shows results for the αA native (αA) and acetyl peptide [αA(a)], and FIG. 1B shows results for the αB native (αB) and acetyl peptide [αB(a)]. We also tested the scrambled αA-crystallin peptide [αA(s)] with two client proteins, CS (peptide: CS-1:1.25) and IN (peptide: IN-1:5); the results are shown in FIG. 1C. The bars represent the mean±SD of three independent assays. *p<0.05, **p<0.005 and ***p<0.0005. NS, not significant.

FIGS. 2(A-H) illustrate crystallin peptides are anti-apoptotic. CHO cells were treated with native (FIGS. 2A and 2C) or acetyl peptides (FIGS. 2B and 2D) in BIOPORTER at 1, 2 and 4 μg/ml medium for 4 h and subjected to thermal stress (43° C. for 1 h) to induce apoptosis. The anti-apoptotic function of the αA-crystallin (FIG. 2E) and αB-crystallin (FIG. 2F) peptides was also assessed in HLE cells. HLE cells were incubated with BP-treated peptides at 4 μg/ml for 4 h and then at 43° C. for 1 h. CHO (FIG. 2G) and HLE cells (FIG. 2 H) were treated with an αA-scrambled peptide at 4 μg/ml to determine peptide specificity. Apoptotic cells were counted after treating the cells with Hoechst stain. The bars represent the mean±SD of three independent experiments. αA, αA-native peptide; αA(a), αA-acetyl peptide; αB, αB-native peptide; αB(a), αB-acetyl peptide; and αA(s), αA-scrambled peptide. **p<0.005 and ***p<0.0005. NS, not significant.

FIGS. 3(A-D) illustrate the inhibition of hyperthermia-induced apoptosis by crystallin peptides occurs through blockade of the mitochondrial death pathway. CHO cells were treated with peptides and thermally stressed. α-crystallin peptides inhibit cytochrome-C release from the mitochondria (A). Cytochrome-C release from the mitochondria into the cytosol was assessed via western blotting. All lanes correspond to thermally stressed cells. Lane 1, no peptide; lane 2, +αA-native peptide; lane 3, +αA-acetyl peptide; lane 4, +αB peptide; and lane 5, +αB-acetyl peptide. Activation of procaspase-3 was inhibited by both acetyl and native peptides (B). Casp-3-FL, full-length procaspase-3; and Active-casp-3, active caspase-3. Lane 1, control; lanes 2 through 6, thermally stressed cells; lane 2, no peptide; lane 3, +αA-native peptide; lane 4, +αA-acetyl peptide; lane 5, +αB peptide; and lane 6, +αB-acetyl peptide. Caspase-3 (C) and caspase-9 (D) activity was measured using specific fluorogenic substrates. Both αA-crystallin and αB-crystallin peptides inhibited caspase activity. The bars represent the mean±SD of three independent experiments. αA, αA-native peptide; αA(a), αA-acetyl peptide; αB, αB-native peptide; and αB(a), αB-acetyl peptide. The differences between the native and acetyl peptides were not statistically significant. **p<0.005, ***p<0.0005.

FIGS. 4(A-D) illustrate a-Crystallin peptides inhibit calcimycin-induced apoptosis in cultured mouse lenses. Mouse lenses were organ cultured and treated with peptides, as described in the Experimental Procedures. After treatment, the lenses were thoroughly rinsed in PBS, fixed, and sectioned. The sections were stained to detect apoptotic cells using an in situ Apoptosis Detection Kit (A). Left panels, DAPI staining to visualize the nuclei in the lens epithelium; right panels, TUNEL staining to show apoptosis (arrows). Panel 1, Control; and panels 2 through 7, calcimycin-treated lenses. Panel 2, no peptide; panel 3, +αA-native peptide; panel 4, +αA-acetyl peptide; panel 5, +αB peptide; panel 6, +αB-acetyl peptide and panel 7, scrambled αA-peptide. The percentages of apoptotic cells are presented in a bar graph format in Panel B. After culturing, the lenses were homogenized, and caspase-3 (C) as well as caspase-9 (D) activity. The bars represent the mean±SD of three independent experiments. αA, αA-native peptide; αA(a), αA-acetyl peptide; αB, αB-native peptide; αB(a), αB-acetyl peptide; and αA(s), αA-scrambled peptide. The differences between the native and acetyl peptides were not statistically significant. The intense TUNEL staining in the nuclear region is likely due to fragmented DNA in the terminally differentiated fiber cells. ***p<0.0005. NS, not significant. Scale bar=100 μm.

FIG. 5 illustrates αA-Crystallin peptides inhibit cataracts in rats. Sodium selenite was administered to 12-day-old rat pups to induce cataracts. When single injections were used, native and acetyl αA-crystallin peptides were i.p. injected at 2.5, 5, and 10 μg/animal 6 h prior to sodium selenite injection. When multiple injections were used, the peptides at the above doses were injected 6 h prior to and on days 1, 2, 3, and 4 days following sodium selenite injection. On day 6-post sodium selenite injection (when the pups were 18 days old and had opened their eyes), cataract formation was assessed via slit-lamp (left panels) and direct imaging (right panels). The control rats exhibited no cataracts (top left), whereas the sodium selenite-injected rats had mature cataracts (top right). The peptides inhibited cataract development; the acetyl peptide (single 5 and 10 μg injections and multiple 2.5 μg injections) was better than the native peptide at inhibiting cataract development. When multiple 10 μg injections were administered, both peptides prevented cataract development. For each treatment group, n=3. The data shown are from one representative animal/group. αA, αA-native peptide; αA(a), αA-acetyl peptide.

FIG. 6 illustrates αB-Crystallin peptides inhibit cataracts in rats. The experimental details are identical to those given in FIG. 5, except that the animals were injected with either the αB native peptide or αB-acetyl peptide. The bottom panels show the results from rats treated with multiple injections (10 μg/animal) of the scrambled αA-crystallin peptide. The native and acetyl αB-crystallin peptides exhibited a similar potency in inhibiting selenite-induced cataracts. In each treatment group, n=3. The data shown are from one representative animal/group. Mild inhibition of cataract was evident following treatment with 5 μg of the acetyl peptide, but not the native peptide. Following multiple injections at 10 μg, both peptides inhibited cataract development. αB, αB-native peptide; αB(a), αB-acetyl peptide; and αA(s), αA-scrambled peptide. The scrambled peptide was ineffective in inhibiting cataract.

FIG. 7 illustrates the inhibition of protein aggregation in selenite-induced cataracts by peptides. Rat pups were treated with multiple injections of peptides (10 μg) as in FIG. 5. After treatment, the lenses were harvested, fixed, sectioned, and stained with H&E. The center of the lens in a selenite cataract is dense (arrow) likely due to protein aggregation, which was inhibited by native and acetyl αA- and αB-crystallin peptides.

FIGS. 8(A-E) illustrate in rats, α-crystallin peptides inhibit protein insolubilization in selenite cataracts. Cataracts were induced using sodium selenite, and the animals were treated with multiple 10 μg peptide injections, as in FIG. 5. The lenses were harvested from animals on day 6-post sodium selenite injection and processed. The water-soluble protein content decreased in the selenite-induced cataracts, which was significantly corrected by the native and acetyl peptides (FIG. 8A). MALS-DLS analyses of the water-soluble protein fraction from control, selenite-treated, and selenite+peptide treated animals showed no apparent differences between groups (FIG. 8B). Water-soluble and insoluble proteins were analyzed by SDS-PAGE, which showed no additional protein crosslinking under either selenite or selenite+peptide treatment (FIGS. 8C and 8D). 1, control; lanes 2 through 6, sodium selenite treated; 2, no peptide; 3, +αA-native peptide; 4, +αA acetyl peptide; 5, +αB peptide; and 6, +αB-acetyl peptide. The cleavage of βB1-crystallin in the water-insoluble selenite cataract fraction (determined by western blotting) was inhibited by peptide administration (FIG. 8E). The bars represent the mean±SD of three independent experiments. The differences between the native and acetyl peptides were not statistically significant.*p<0.05, **p<0.005, and ***p<0.0005. NS, not significant.

FIGS. 9(A-B) illustrate α-crystallin peptides inhibit oxidative stress in selenite-induced cataracts. The lenses were harvested from animals on day 6-post sodium selenite injection and processed as described in the Experimental Procedures. α-crystallin peptides (both acetyl and native peptides) administered through multiple injections of 10 μg/animal (as in FIG. 5) inhibited the loss of GSH (FIG. 9A) and SOD1 activity in selenite-treated rat lenses (FIG. 9B). The bars represent the mean±SD of three independent experiments. αA, αA-native peptide; αA(a), αA-acetyl peptide; αB, αB-native peptide; and αB(a), αB-acetyl peptide. The differences between native and acetyl peptides were not statistically significant. *p<0.05, **p<0.005, and ***p<0.0005. NS, not significant.

FIGS. 10(A-C) illustrate α-crystallin peptides inhibit apoptosis through inhibition of caspases in lenses with selenite-induced cataract. The lenses from sodium selenite- and sodium selenite+peptide-treated rat pups (multiple 10 μg peptide injections, as in FIG. 5) were fixed and assessed for epithelial cell apoptosis using the in situ Cell Death Detection Kit (FIG. 10A). DAPI staining is shown in the left panels, and TUNEL staining is shown on the right. 1, control; lanes 2 through 6, sodium selenite treated; 2, no peptide, arrows indicate apoptotic cells; 3, +αA-native peptide; 4, +αA-acetyl peptide; 5, +αB peptide; 6, +αB-acetyl peptide; and 7, +αA scrambled peptide. The enlarged images for A2 and A7 are to show apoptotic cells. The elevation of caspase-3 and caspase-9 activity observed in selenite-induced cataracts was inhibited by both the native and acetyl αA- and αB-crystallin peptides (FIGS. 10B and 10C). The bars represent the mean±SD of three independent experiments. αA, αA-native peptide; αA(a), αA-acetyl peptide; αB, αB-native peptide; and αB(a), αB-acetyl peptide. The differences between the native and acetyl peptides were not statistically significant. *p<0.05, **p<0.005, and ***p<0.0005. NS, not significant. Scale bar=100 μm.

FIGS. 11(A-C) illustrate I.p.-injected αB-acetyl crystallin peptide translocates to the lens in rats. Rats were administered 100 μg of the αB-acetyl peptide in water i.p. for five days. The lenses were then harvested and homogenized in 500 μl of buffer with 6 M urea. The homogenate was filtered through a 10 kDa cut-off centrifugal filter, and the filtrate was analyzed via LCMS/MS. FIG. 11A shows the base peak ion chromatogram for the injected sample. The arrow indicates the peak of αB-acetyl crystallin peptide. The full mass spectrum in FIG. 11B shows the triply and quadruply charged parent ions at m/z 843.4560(3+) and 632.8441(4+) for the peptide. K92 acetylation in the peptide (SEQ ID NO: 14) was confirmed by MS/MS analysis of the two parent ions. The MS/MS spectrum for the triply charged ion is shown in FIG. 11C. The peptide sequence was assigned based on the fragmented y and b ions.

### DETAILED DESCRIPTION

The embodiments described herein are not limited to the particular methodology, protocols, and reagents, etc., and as such may vary. The terminology used herein is for the purpose of describing particular embodiments only, and is not intended to limit the scope of the present invention, which is defined solely by the claims. Other than in the operating examples, or where otherwise indicated, all numbers expressing quantities of ingredients or reaction conditions used herein should be understood as modified in all instances by the term “about.”

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and does not constitute any admission as to the correctness of the dates or contents of these documents.

Unless otherwise defined, scientific and technical terms used herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art.

As used herein, “one or more of a, b, and c” means a, b, c, ab, ac, bc, or abc. The use of “or” herein is the inclusive or.

As used herein, the term “administering” to a patient includes dispensing, delivering or applying an active compound in a pharmaceutical formulation to a subject by any suitable route for delivery of the active compound to the desired location in the subject (e.g., to thereby contact a desired cell such as a desired epithelial cell), including administration into the cerebrospinal fluid or across the blood-brain barrier, delivery by either the parenteral or oral route, intramuscular injection, subcutaneous or intradermal injection, intravenous injection, buccal administration, transdermal delivery and administration by the rectal, colonic, vaginal, intranasal or respiratory tract route. The compositions or agents may, for example, be administered to a comatose, anesthetized or paralyzed subject via an intravenous injection or may be administered intravenously to a pregnant subject to stimulate axonal growth in a fetus. Specific routes of administration may include topical application, such as by eyedrops, creams or erodible formulations to be placed under the eyelid, intraocular injection into the aqueous or the vitreous humor, injection into the external layers of the eye, such as via subconjunctival injection or subtenon injection, parenteral administration or via oral routes.

As used herein, a “chimeric protein” or “fusion protein” is a fusion of a first amino acid sequence encoding a polypeptide with a second amino acid sequence defining a domain (e.g., polypeptide portion) foreign to and not substantially homologous with the domain of the first polypeptide. A chimeric protein may present a foreign domain, which is found (albeit in a different protein) in an organism, which also expresses the first protein, or it may be an “interspecies”, “intergenic”, etc. fusion of protein structures expressed by different kinds of organisms.

As used herein, the term “contacting cell” or “treating cells” refers to any mode of agent delivery or “administration,” either to cells or to whole organisms, in which the agent is capable of exhibiting its pharmacological effect in the cells. “Contacting cells” includes both in vivo and in vitro methods of bringing an agent of the invention into proximity with a neuron. Suitable modes of administration can be determined by those skilled in the art and such modes of administration may vary between agents. For example, when apoptosis is mitigated, agents can be administered, for example, by transfection, lipofection, electroporation, viral vector infection, or by addition to growth medium.

As used herein an “effective amount” of an agent or therapeutic peptide is an amount sufficient to achieve a desired therapeutic or pharmacological effect, such as an amount that is capable of activating the growth of neurons. An effective amount of an agent as defined herein may vary according to factors such as the disease state, age, and weight of the subject, and the ability of the agent to elicit a desired response in the subject. Dosage regimens may be adjusted to provide the optimum therapeutic response. An effective amount is also one in which any toxic or detrimental effects of the active compound are outweighed by the therapeutically beneficial effects.

As used herein, the term a “therapeutically effective amount” refers to an amount effective, at dosages and for periods of time necessary, to achieve the desired therapeutic result. A therapeutic result may be, e.g., lessening of symptoms, prolonged survival, improved mobility, and the like. A therapeutic result need not be a “cure.”

As used herein, the term “expression” refers to the process by which nucleic acid is translated into peptides or is transcribed into RNA, which, for example, can be translated into peptides, polypeptides or proteins. If the nucleic acid is derived from genomic DNA, expression may, if an appropriate eukaryotic host cell or organism is selected, include splicing of the mRNA. For heterologous nucleic acid to be expressed in a host cell, it must initially be delivered into the cell and then, once in the cell, ultimately reside in the nucleus.

As used herein, the term “genetic therapy” involves the transfer of heterologous DNA to cells of a mammal, particularly a human, with a disorder or conditions for which therapy or diagnosis is sought. The DNA is introduced into the selected target cells in a manner such that the heterologous DNA is expressed and a therapeutic product encoded thereby is produced. Alternatively, the heterologous DNA may in some manner mediate expression of DNA that encodes the therapeutic product; it may encode a product, such as a peptide or RNA that in some manner mediates, directly or indirectly, expression of a therapeutic product. Genetic therapy may also be used to deliver nucleic acid encoding a gene product to replace a defective gene or supplement a gene product produced by the mammal or the cell in which it is introduced. The introduced nucleic acid may encode a therapeutic compound, such as a growth factor inhibitor thereof, or a tumor necrosis factor or inhibitor thereof, such as a receptor therefore, that is not normally produced in the mammalian host or that is not produced in therapeutically effective amounts or at a therapeutically useful time. The heterologous DNA encoding the therapeutic product may be modified prior to introduction into the cells of the afflicted host in order to enhance or otherwise alter the product or expression thereof.

As used herein, the term “gene” or “recombinant gene” refers to a nucleic acid comprising an open reading frame encoding a polypeptide, including both exon and (optionally) intron sequences.

As used herein, the term “heterologous nucleic acid sequence” is typically DNA that encodes RNA and proteins that are not normally produced in vivo by the cell in which it is expressed or that mediates or encodes mediators that alter expression of endogenous DNA by affecting transcription, translation, or other regulatable biochemical processes. A heterologous nucleic acid sequence may also be referred to as foreign DNA. Any DNA that one of skill in the art would recognize or consider as heterologous or foreign to the cell in which it is expressed is herein encompassed by heterologous DNA. Antibodies that are encoded by heterologous DNA may be secreted or expressed on the surface of the cell in which the heterologous DNA has been introduced.

As use herein, the terms “homology” and “identity” are used synonymously throughout and refer to sequence similarity between two peptides or between two nucleic acid molecules. Homology can be determined by comparing a position in each sequence, which may be aligned for purposes of comparison. When a position in the compared sequence is occupied by the same base or amino acid, then the molecules are homologous or identical at that position. A degree of homology or identity between sequences is a function of the number of matching or homologous positions shared by the sequences.

As used herein, the phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, subcapsular, subarachnoid, intraspinal and intrasternal injection and infusion.

As used herein, the phrases “systemic administration,” “administered systemically,” “peripheral administration” and “administered peripherally” as used herein mean the administration of a compound, drug or other material other than directly into a target tissue (e.g., the central nervous system), such that it enters the animal's system and, thus, is subject to metabolism and other like processes, for example, subcutaneous administration.

As use herein, the term “patient” or “subject” or “animal” or “host” refers to any mammal. The subject may be a human, but can also be a mammal in need of veterinary treatment, e.g., domestic animals (e.g., dogs, cats, and the like), farm animals (e.g., cows, sheep, fowl, pigs, horses, and the like) and laboratory animals (e.g., rats, mice, guinea pigs, and the like).

As used herein, the terms “polynucleotide sequence” and “nucleotide sequence” are also used interchangeably herein.

As used herein, the terms “peptide” or “polypeptide” are used interchangeably herein and refer to compounds consisting of from about 2 to about 90 amino acid residues, inclusive, wherein the amino group of one amino acid is linked to the carboxyl group of another amino acid by a peptide bond. A peptide can be, for example, derived or removed from a native protein by enzymatic or chemical cleavage, or can be prepared using conventional peptide synthesis techniques (e.g., solid phase synthesis) or molecular biology techniques (see Sambrook et al., MOLECULAR CLONING: LAB. MANUAL (Cold Spring Harbor Press, Cold Spring Harbor, N.Y., 1989)). A “peptide” can comprise any suitable L- and/or D-amino acid, for example, common a-amino acids (e.g., alanine, glycine, valine), non-a-amino acids (e.g., P-alanine, 4-aminobutyric acid, 6aminocaproic acid, sarcosine, statine), and unusual amino acids (e.g., citrulline, homocitruline, homoserine, norleucine, norvaline, ornithine). The amino, carboxyl and/or other functional groups on a peptide can be free (e.g., unmodified) or protected with a suitable protecting group. Suitable protecting groups for amino and carboxyl groups, and means for adding or removing protecting groups are known in the art. See, e.g., Green &amp; Wuts, PROTECTING GROUPS IN ORGANIC SYNTHESIS (John Wiley &amp; Sons, 1991). The functional groups of a peptide can also be derivatized (e.g., alkylated) using art-known methods.

As used herein, the term “peptidomimetic”, refers to a protein-like molecule designed to mimic a peptide. Peptidomimetics typically arise either from modification of an existing peptide, or by designing similar systems that mimic peptides, such as peptoids and β-peptides. Irrespective of the approach, the altered chemical structure is designed to advantageously adjust the molecular properties such as, stability or biological activity. These modifications involve changes to the peptide that do not occur naturally (such as altered backbones and the incorporation of nonnatural amino acids).

A polynucleotide sequence (DNA, RNA) is “operatively linked” to an expression control sequence when the expression control sequence controls and regulates the transcription and translation of that polynucleotide sequence. The term “operatively linked” includes having an appropriate start signal (e.g., ATG) in front of the polynucleotide sequence to be expressed, and maintaining the correct reading frame to permit expression of the polynucleotide sequence under the control of the expression control sequence, and production of the desired polypeptide encoded by the polynucleotide sequence.

As used herein, the term “recombinant,” as used herein, means that a protein is derived from a prokaryotic or eukaryotic expression system.

As used herein, the term “tissue-specific promoter” means a nucleic acid sequence that serves as a promoter, i.e., regulates expression of a selected nucleic acid sequence operably linked to the promoter, and which affects expression of the selected nucleic acid sequence in specific cells of a tissue. The term also covers so-called “leaky” promoters, which regulate expression of a selected nucleic acid primarily in one tissue, but cause expression in other tissues as well. The term “transfection” is used to refer to the uptake of foreign DNA by a cell. A cell has been “transfected” when exogenous DNA has been introduced inside the cell membrane. A number of transfection techniques are generally known in the art. See, e.g., Graham et al., Virology 52:456 (1973); Sambrook et al., Molecular Cloning: A Laboratory Manual (1989); Davis et al., Basic Methods in Molecular Biology (1986); Chu et al., Gene 13:197 (1981). Such techniques can be used to introduce one or more exogenous DNA moieties, such as a nucleotide integration vector and other nucleic acid molecules, into suitable host cells. The term captures chemical, electrical, and viral-mediated transfection procedures.

As used herein, the terms “transcriptional regulatory sequence” is a generic term used throughout the specification to refer to nucleic acid sequences, such as initiation signals, enhancers, and promoters, which induce or control transcription of protein coding sequences with which they are operably linked. In some examples, transcription of a recombinant gene is under the control of a promoter sequence (or other transcriptional regulatory sequence), which controls the expression of the recombinant gene in a cell-type in which expression is intended. It will also be understood that the recombinant gene can be under the control of transcriptional regulatory sequences which are the same or which are different from those sequences, which control transcription of the naturally occurring form of a protein.

As used herein, the term “vector” refers to a nucleic acid molecule capable of transporting another nucleic acid to which it has been linked. Preferred vectors are those capable of one or more of, autonomous replication and expression of nucleic acids to which they are linked. Vectors capable of directing the expression of genes to which they are operatively linked are referred to herein as “expression vectors”.

As used herein, the term “wild type” refers to the naturally-occurring polynucleotide sequence encoding a protein, or a portion thereof, or protein sequence, or portion thereof, respectively, as it normally exists in vivo. As used herein, the term “nucleic acid” refers to polynucleotides, such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

The agents, compounds, compositions, antibodies, etc. used in the methods described herein are considered to be purified and/or isolated prior to their use. Purified materials are typically “substantially pure”, meaning that a nucleic acid, polypeptide or fragment thereof, or other molecule has been separated from the components that naturally accompany it. Typically, the polypeptide is substantially pure when it is at least 60%, 70%, 80%, 90%, 95%, or even 99%, by weight, free from the proteins and other organic molecules with which it is associated naturally. For example, a substantially pure polypeptide may be obtained by extraction from a natural source, by expression of a recombinant nucleic acid in a cell that does not normally express that protein, or by chemical synthesis. “Isolated materials” have been removed from their natural location and environment. In the case of an isolated or purified domain or protein fragment, the domain or fragment is substantially free from amino acid sequences that flank the protein in the naturally-occurring sequence. The term “isolated DNA” means DNA has been substantially freed of the genes that flank the given DNA in the naturally occurring genome. Thus, the term “isolated DNA” encompasses, for example, cDNA, cloned genomic DNA, and synthetic DNA.

As used herein, the terms “portion”, “fragment”, “variant”, “derivative” and “analog”, when referring to a polypeptide of the present invention include any polypeptide that retains at least some biological activity referred to herein (e.g., inhibition of an interaction such as binding). Polypeptides as described herein may include portion, fragment, variant, or derivative molecules without limitation, as long as the polypeptide still serves its function. Polypeptides or portions thereof of the present invention may include proteolytic fragments, deletion fragments and in particular, or fragments that more easily reach the site of action when delivered to an animal.

Embodiments described herein relate to compositions and methods of inhibiting, reducing, and/or treating pathological apoptosis and/or protein aggregation and to methods and compositions of treating diseases, disorders, and/or conditions associated with pathological apoptosis and/or pathological protein aggregation. The compositions include therapeutic polypeptides (or proteins) that can inhibit pathological protein aggregation and/or pathological apoptosis in cells, such as neural cells, ganglion cells, and epithelial cells. The therapeutic polypeptides can include acetylated αA-crystallin, acetylated αB-crystallin, acetylated fragments thereof having molecular chaperone activity, and/or polypeptide mimetics thereof that can inhibit pathological protein aggregation and/or pathological apoptosis.

It was found that acetylated αA-crystallin, acetylated αB-crystallin, acetylated polypeptide fragments thereof having molecular chaperone activity, and polypeptide mimetics thereof showed 10-40% increase in the chaperone function in comparison to unacetylated αA-crystallin, unacetylated αB-crystallin, and unacetylated polypeptide fragments thereof having molecular chaperone activity. Acetylated αA-crystallin, acetylated αB-crystallin, acetylated polypeptide fragments, and polypeptide mimetic thereof also showed greater capacity to inhibit apoptosis in thermally stressed Chinese hamster ovary cells and human lens epithelial cells and higher inhibitory capacity against in vitro activation of capase-3 and 9 than unacetylated αA-crystallin, unacetylated αB-crystallin, and unacetylated polypeptide fragments. The calcimycin-induced apoptosis of epithelial cells in organ cultured rat lenses was inhibited to a greater extent by acetylated αA-crystallin, acetylated αB-crystallin, acetylated polypeptide fragments thereof, and polypeptide mimetics thereof than unacetylated αA-crystallin, unacetylated αB-crystallin, and unacetylated polypeptide fragments. Intraperitoneal injection of acetylated αA-crystallin, acetylated αB-crystallin, and acetylated polypeptide fragments thereof, and polypeptide mimetics thereof dose-dependently inhibited lens protein aggregation, insolubilization and cataract development in selenite-treated rat pups. These effects were accompanied by inhibition of oxidative stress, caspase activities and apoptosis of lens epithelial cells.

Moreover, it was found that intraperitoneally injected acetylated polypeptide fragments of αA-crystallin and αB-crystallin and polypeptide mimetics thereof can cross the blood aqueous barrier and cell plasma membrane and treat pathogenic apoptosis associated with eye diseases, such as age-related macular degeneration, uveitis, glaucoma, and diabetic retinopathy. Because the polypeptides can cross the blood retinal barrier, they are useful for inhibiting apoptosis associated with diseases. Furthermore, when apoptosis in cells is accompanied by protein aggregation, such as in Alzheimer's disease, the polypeptides can advantageously inhibit both protein aggregation and apoptosis in cells.

In some embodiments, the acetylated αA-crystallin and acetylated αB-crystallin include at least one lysine that is acetylated. The acetylated lysine can be, for example, K70 of αA-crystallin and K92 of αB-crystallin. The acetylated αA-crystallin can have an amino acid sequence with a sequence identity of SEQ ID NO: 1; and the acetylated αB-crystallin can have an amino acid sequence with a sequence identity of SEQ ID NO: 2.

In other embodiments, the acetylated polypeptide fragments of αA-crystallin and/or αB-crystallin having molecular chaperone activity can include polypeptides that consist of about 18 to about 25 amino acid and that have an at least 90% or at least 95% sequence identity to 18 to 25 consecutive amino acids of molecular chaperone portions of αA-crystallin and/or αB-crystallin that include acetylated K70 of αA-crystallin or acetylated K92 of αB-crystallin. For example, the acetylated polypeptide fragment of αA-crystallin can have an amino acid sequence with a sequence identity of SEQ ID NO: 3, and the acetylated polypeptide fragments of αB-crystallin can have an amino acid sequence with a sequence identity of SEQ ID NO: 4.

The polypeptide mimetics of the acetylated αA-crystallin, acetylated αB-crystallin, and acetylated polypeptide fragments thereof can have an amino acid sequence that is the same or at least 90% or at least 95% the same as the amino acid sequence of the acetylated αA-crystallin, acetylated αB-crystallin, and acetylated polypeptide fragments thereof except the polypeptide mimetic includes a glutamine residue instead of an acetylated lysine. In some embodiments, the polypeptide mimetics of the acetylated αA-crystallin, acetylated αB-crystallin, and acetylated polypeptide fragments thereof can have amino acid sequences with sequence identities of SEQ ID NO: 5, SEQ ID NO: 6, and SEQ ID NO: 7, and SEQ ID NO: 8.

In other embodiments, the acetylated αA-crystallin, acetylated αB-crystallin, acetylated polypeptide fragments thereof, and/or polypeptide mimetics thereof can have an amino acid sequence with a sequence identity selected from the group consisting of SEQ ID NO: 9, SEQ ID NO: 10, SEQ ID NO: 11 and SEQ ID NO: 12, wherein X is an acetylated lysine or glutamine.

The therapeutic polypeptides described herein may be prepared by methods known to those skilled in the art. The polypeptides may be prepared using recombinant DNA. For example, one preparation can include cultivating a host cell (bacterial or eukaryotic) under conditions, which provide for the expression of peptides and/or proteins within the cell.

The purification of the polypeptides may be done by affinity methods, ion exchange chromatography, size exclusion chromatography, hydrophobicity or other purification technique typically used for protein purification. The purification step can be performed under non-denaturating conditions. On the other hand, if a denaturating step is required, the protein may be renatured using techniques known in the art.

In some embodiments, the therapeutic polypeptides described herein can include additional residues that may be added at either terminus of a polypeptide for the purpose of providing a “linker” by which the polypeptides can be conveniently linked and/or affixed to other polypeptides, proteins, detectable moieties, labels, solid matrices, or carriers.

Amino acid residue linkers are usually at least one residue and can be 40 or more residues, more often 1 to 10 residues. Typical amino acid residues used for linking are glycine, tyrosine, cysteine, lysine, glutamic and aspartic acid, or the like. In addition, a subject polypeptide can differ by the sequence being modified by terminal-NH2 acylation, e.g., acetylation, or thioglycolic acid amidation, by terminal-carboxylamidation, e.g., with ammonia, methylamine, and the like terminal modifications. Terminal modifications are useful, as is well known, to reduce susceptibility by proteinase digestion, and therefore serve to prolong half life of the polypeptides in solutions, particularly biological fluids where proteases may be present. In this regard, polypeptide cyclization is also a useful terminal modification, and is particularly preferred also because of the stable structures formed by cyclization and in view of the biological activities observed for such cyclic peptides as described herein.

In some embodiments, the linker can be a flexible peptide linker that links the therapeutic polypeptide to other polypeptides, proteins, and/or molecules, such as detectable moieties, labels, solid matrices, or carriers. A flexible peptide linker can be about 20 or fewer amino acids in length. For example, a peptide linker can contain about 12 or fewer amino acid residues, e.g., 3, 4, 5, 6, 7, 8, 9, 10, 11, and 12. In some cases, a peptide linker comprises two or more of the following amino acids: glycine, serine, alanine, and threonine.

In some embodiments, a composition comprising the therapeutic polypeptides described herein can be provided with a carrier reagent that can facilitate transport of the therapeutic polypeptides into a cell. Carrier reagents may comprise a variety of species. In one embodiment, the carrier reagent is a bioactive cell membrane-permeable reagent, or other peptides containing protein-transduction domains (PTDs) (i.e., single peptide sequences comprising about 15 to about 30 residues). Protein-transduction domains (PTDs) mediate protein secretion, and are composed of a positively charged amino terminus, a central hydrophobic core and a carboxyl-terminal cleavage site recognized by a single peptidase.

The PTDs can be covalently linked to the therapeutic polypeptides. The covalent link can include a peptide bond or a labile bond (e.g., a bond readily cleavable or subject to chemical change in the interior target cell environment). Additionally, the PTDs can be cross-linked (e.g., chemically cross-linked, UV cross-linked) to the therapeutic polypeptide. The PTDs can also be linked to the therapeutic polypeptide with linking polypeptide described herein.

The PTDs can be repeated more than once in the composition comprising the therapeutic polypeptide. The repetition of a PTD may affect (e.g., increase) the uptake of the polypeptides by a desired cell. The PTDs may also be located either at the amino-terminal region of therapeutic peptide or at its carboxy-terminal region or at both regions.

In one embodiment, the PTDs can include at least one transport peptide sequence that allows the therapeutic polypeptide once linked to the transport moiety to penetrate into the cell by a receptor-independent mechanism. In one example, the transport peptide is a synthetic peptide that contains a Tat-mediated protein delivery sequence.

Other examples of known PTDs, subdomains and the like are described in, for example, Canadian patent document No. 2,301,157 (conjugates containing homeodomain of antennapedia) as well as in U.S. Pat. Nos. 5,652,122, 5,670,617, 5,674,980, 5,747,641, and 5,804,604, all of which are incorporated herein by reference in their entirety, (conjugates containing amino acids of Tat HIV protein; herpes simplex virus-1 DNA binding protein VP22, a Histidine tag ranging in length from 4 to 30 histidine repeats, or a variation derivative or homologue thereof capable of facilitating uptake of the active cargo moiety by a receptor independent process.

A 16 amino acid region of the third alpha-helix of antennapedia homeodomain has also been shown to enable proteins (made as fusion proteins) to cross cellular membranes (PCT international publication number WO 99/11809 and Canadian application No.: 2,301,157. Similarly, HIV Tat protein was shown to be able to cross cellular membranes.

In addition, the PTDs can include polypeptides having a basic amino acid rich region covalently linked to an active agent moiety (e.g., intracellular domain-containing fragments inhibitor peptide). As used herein, the term “basic amino acid rich region” relates to a region of a protein with a high content of the basic amino acids such as arginine, histidine, asparagine, glutamine, lysine. A “basic amino acid rich region” may have, for example 15% or more of basic amino acid. In some instance, a “basic amino acid rich region” may have less than 15% of basic amino acids and still function as a transport agent region. In other instances, a basic amino acid region will have 30% or more of basic amino acids.

The transport moiety(ies) may further include a proline rich region. As used herein, the term proline rich region refers to a region of a polypeptide with 5% or more (up to 100%) of proline in its sequence. In some instance, a proline rich region may have between 5% and 15% of prolines. Additionally, a proline rich region refers to a region, of a polypeptide containing more prolines than what is generally observed in naturally occurring proteins (e.g., proteins encoded by the human genome). Proline rich regions of this application can function as a transport agent region.

In one embodiment, the therapeutic polypeptide described herein can be non-covalently linked to a transduction agent. An example of a non-covalently linked polypeptide transduction agent is the Chariot protein delivery system (See U.S. Pat. No. 6,841,535; J Biol Chem 274(35):24941-24946; and Nature Biotec. 19:1173-1176, all herein incorporated by reference in their entirety).

In another embodiment, the carrier reagent is a lipid liposome or the like that can complex with the therapeutic polypeptide and promote the delivery of the therapeutic polypeptide into the cell. For example, the polypeptide encapsulated in the formulation binds to the negatively vehicle for delivery (O. Zelphati et al., J. Bio. Chem., 276, 35103-19 (2001)). Products available commercially can be used, such as BioPORTER (Gene Therapy Systems), or ProVectin (Imgenex, San Diego, Calif.).

Protein delivery reagents (e.g., CHARIOT by Active Motif, or BIOPORTER by Gene Therapy Systems) can help save time by bypassing the traditional DNA transfection, transcription and protein translation processes associated with gene expression. Depending on the nature of the particular reagent employed, fusion proteins or chemical coupling in some embodiments would not be needed. The reagent forms a complex with the protein, stabilizes the macromolecule and protects it from degradation during delivery. Once internalized in a cell, the complex can dissociate, leaving the macromolecule biologically active and free to proceed to its target organelle.

The therapeutic polypeptides described herein may further be modified (e.g., chemically modified). Such modification may be designed to facilitate manipulation or purification of the molecule, to increase solubility of the molecule, to facilitate administration, targeting to the desired location, to increase or decrease half life. A number of such modifications are known in the art and can be applied by the skilled practitioner.

The compositions comprising the therapeutic polypeptides can be delivered to cells in vivo or in vitro by directly contacting the therapeutic polypeptide with specific cell surface receptors, injecting into the cell, or by means of any delivery tag, carrier, vehicle, or technique known and suitable in the art, including a liposome, micelle, fusion tag, antibody, carrier protein, chemical moiety, electroporation, microinjection, viral protein fusions, nanoparticles, and commercial protein delivery reagents. The therapeutic polypeptides can be internalized into the cell, preferably into the cytoplasm, by any passive, facilitated, or active processes.

When the compositions comprising the therapeutic polypeptides are delivered to a subject, they can be administered by any suitable route, including, for example, orally (e.g., in capsules, suspensions or tablets), systemically, or by parenteral administration. Parenteral administration can include, for example, intramuscular, intravenous, intraarticular, intraarterial, intrathecal, subcutaneous, or intraperitoneal administration. The composition can also be administered orally, transdermally, topically, by inhalation (e.g., intrabronchial, intranasal, oral inhalation or intranasal drops) or rectally. Administration can be local or systemic as indicated.

Both local and systemic administration are contemplated herein. Desirable features of local administration include achieving effective local concentrations of the therapeutic polypeptide as well as avoiding adverse side effects from systemic administration of the therapeutic polypeptide. In one embodiment, a composition comprising the therapeutic polypeptide can be administered by introduction into the retina of the subject. In another aspect, a composition comprising the therapeutic polypeptide can be introduced locally, such as into the site of nerve or cord injury, into a site of neural degeneration, or intraocularly to contact neuroretinal cells.

In another embodiment, compositions comprising the therapeutic polypeptides can be administered into a subject intrathecally. As used herein, the term “intrathecal administration” is intended to include delivering a composition comprising the therapeutic polypeptide directly into the cerebrospinal fluid of a subject, by techniques including lateral cerebroventricular injection through a burrhole or cisternal or lumbar puncture or the like (described in Lazorthes et al., 1991, and Ommaya, 1984, the contents of which are incorporated herein by reference). The term “lumbar region” is intended to include the area between the third and fourth lumbar (lower back) vertebrae. The term “cistema magna” is intended to include the area where the skull ends and the spinal cord begins at the back of the head. The ten-n “cerebral ventricle” is intended to include the cavities in the brain that are continuous with the central canal of the spinal cord. Administration of the composition described herein to any of the above mentioned sites can be achieved by direct injection of the therapeutic agent or by the use of infusion pumps. Implantable or external pumps and catheter may be used.

For injection, the composition can be formulated in liquid solutions, typically in physiologically compatible buffers such as Hank's solution or Ringer's solution. In addition, the therapeutic agent may be formulated in solid form and re-dissolved or suspended immediately prior to use. Lyophilized forms are also included. The injection can be, for example, in the form of a bolus injection or continuous infusion (such as using infusion pumps) of the therapeutic agent.

In one embodiment, the composition comprising the therapeutic polypeptide can be administered by lateral cerebroventricular injection into the brain of a subject, usually within 100 hours of when an injury occurs (such as within 6, 12, 24 or 100 hours, inclusive, from the time of the injury). The injection can be made, for example, through a burr hole made in the subject's skull. In another embodiment, the composition comprising the therapeutic polypeptide can be administered through a surgically inserted shunt into the cerebral ventricle of a subject, usually within 100 hours of when an injury occurs (e.g., within 6, 12 or 24 hours, inclusive, from the time of the injury). For example, the injection can be made into the lateral ventricles, which are larger, even though injection into the third and fourth smaller ventricles can also be made. In yet another embodiment, the therapeutic agent can be administered by injection into the cistema magna, or lumbar area of a subject, within 100 hours of when an injury occurs (such as within 6, 12, or 24 hours, inclusive, from the time of the injury).

An additional means of administration to intracranial tissue involves application to the olfactory epithelium, with subsequent transmission to the olfactory bulb and transport to more proximal portions of the brain. Such administration can be by nebulized or aerosolized preparations.

In another embodiment, the therapeutic polypeptide can be administered to a subject at the site of injury, usually within 100 hours of when an injury occurs (e.g., within 6, 12, or 24 hours, inclusive, of the time of the injury).

In a further embodiment, ophthalmic compositions of the therapeutic polypeptides described herein can also be used to prevent or reduce damage to retinal and optic nerve head tissues, as well as to enhance functional recovery after damage to ocular tissues. Ophthalmic conditions that may be treated include, but are not limited to, retinopathies (including diabetic retinopathy and retrolental fibroplasia), macular degeneration, ocular ischemia, glaucoma, and cataracts. Other conditions to be treated with the methods of the invention include damage associated with injuries to ophthalmic tissues, such as ischemia reperfusion injuries, photochemical injuries, and injuries associated with ocular surgery, particularly injuries to the retina or optic nerve head by exposure to light or surgical instruments. The ophthalmic compositions may include a pharmaceutically acceptable carrier and can be used as an adjunct to ophthalmic surgery, such as by vitreal or subconjunctival injection following ophthalmic surgery. The therapeutic agents may be used for acute treatment of temporary conditions, or may be administered chronically, especially in the case of degenerative disease. The ophthalmic compositions may also be used prophylactically, especially prior to ocular surgery or noninvasive ophthalmic procedures or other types of surgery.

Formulation of pharmaceutical compounds for use in the modes of administration noted above (and others) are described, for example, in Remington's Pharmaceutical Sciences (18th edition), ed. A. Gennaro, 1990, Mack Publishing Company, Easton, Pa. (also see, e.g., M. J. Rathbone, ed., Oral Mucosal Drug Delivery, Drugs and the Pharmaceutical Sciences Series, Marcel Dekker, Inc., N.Y., U.S.A., 1996; M. J. Rathbone et al., eds., Modified-Release Drug Delivery Technology, Drugs and the Pharmaceutical Sciences Series, Marcel Dekker, Inc., N.Y., U.S.A., 2003; Ghosh et al., eds., Drug Delivery to the Oral Cavity, Drugs and the Pharmaceutical Sciences Series, Marcel Dekker, Inc., N.Y. U.S.A., 1999.

In one example, the therapeutic polypeptide can be provided in ophthalmic preparation that can be administered to the subject's eye. The ophthalmic preparation can contain the therapeutic polypeptide in a pharmaceutically acceptable solution, suspension or ointment. Some variations in concentration will necessarily occur, depending on the particular primary amine compound employed, the condition of the subject to be treated and the like, and the person responsible for treatment will determine the most suitable concentration for the individual subject. The ophthalmic preparation can be in the form of a sterile aqueous solution containing, if desired, additional ingredients, for example, preservatives, buffers, tonicity agents, antioxidants, stabilizers, nonionic wetting or clarifying agents, and viscosity increasing agents.

Examples of preservatives for use in such a solution include benzalkonium chloride, benzethonium chloride, chlorobutanol, thimerosal and the like. Examples of buffers include boric acid, sodium and potassium bicarbonate, sodium and potassium borates, sodium and potassium carbonate, sodium acetate, and sodium biphosphate, in amounts sufficient to maintain the pH at between about pH 6 and about pH 8, and for example, between about pH 7 and about pH 7.5. Examples of tonicity agents are dextran 40, dextran 70, dextrose, glycerin, potassium chloride, propylene glycol, and sodium chloride.

Examples of antioxidants and stabilizers include sodium bisulfite, sodium metabisulfite, sodium thiosulfite, and thiourea. Examples of wetting and clarifying agents include polysorbate 80, polysorbate 20, poloxamer 282 and tyloxapol. Examples of viscosity-increasing agents include gelatin, glycerin, hydroxyethylcellulose, hydroxmethylpropylcellulose, lanolin, methylcellulose, petrolatum, polyethylene glycol, polyvinyl alcohol, polyvinylpyrrolidone, and carboxymethylcellulose. The ophthalmic preparation will be administered topically to the eye of the subject in need of treatment by conventional methods, for example, in the form of drops or by bathing the eye in the ophthalmic solution.

In some embodiments, compositions comprising the therapeutic polypeptides can be administered to a subject for an extended period of time to mitigate or inhibit cell apoptosis. Sustained contact with the active compound can be achieved, for example, by repeated administration of the active compound(s) over a period of time, such as one week, several weeks, one month or longer. The pharmaceutically acceptable formulation used to administer the therapeutic agent(s) can also be formulated to provide sustained delivery of the active compound to a subject. For example, the formulation may deliver the active compound for at least one, two, three, or four weeks, inclusive, following initial administration to the subject. For example, a subject to be treated in accordance with the present invention is treated with the active compound for at least 30 days (either by repeated administration or by use of a sustained delivery system, or both).

Sustained delivery of the therapeutic polypeptide can be demonstrated by, for example, the continued therapeutic effect of the therapeutic polypeptide over time. Alternatively, sustained delivery of the therapeutic agent may be demonstrated by detecting the presence of the therapeutic agents in vivo over time.

Approaches for sustained delivery include use of a polymeric capsule, a minipump to deliver the formulation, a biodegradable implant, or implanted transgenic autologous cells (see U.S. Pat. No. 6,214,622). Implantable infusion pump systems (e.g., INFUSAID pumps (Towanda, Pa.)); see Zierski et al., 1988; Kanoff, 1994) and osmotic pumps (sold by Alza Corporation) are available commercially and otherwise known in the art. Another mode of administration is via an implantable, externally programmable infusion pump. Infusion pump systems and reservoir systems are also described in, e.g., U.S. Pat. No. 5,368,562 and No. 4,731,058.

In other embodiments, polypeptide mimetics of the acetylated αA-crystallin, acetylated αB-crystallin, and acetylated polypeptide fragments thereof the can be expressed in cells being treated using gene therapy. The gene therapy can use a vector including a nucleotide encoding the polypeptide mimetics. A “vector” (sometimes referred to as gene delivery or gene transfer “vehicle”) refers to a macromolecule or complex of molecules comprising a polynucleotide to be delivered to the cell. The polynucleotide to be delivered may comprise a coding sequence of interest in gene therapy. Vectors include, for example, viral vectors (such as adenoviruses (Ad), adeno-associated viruses (AAV), and retroviruses), liposomes and other lipid-containing complexes, and other macromolecular complexes capable of mediating delivery of a polynucleotide to a target cell.

Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities.

Selectable markers can be positive, negative or bifunctional. Positive selectable markers allow selection for cells carrying the marker, whereas negative selectable markers allow cells carrying the marker to be selectively eliminated. A variety of such marker genes have been described, including bifunctional (i.e., positive/negative) markers (see, e.g., Lupton, S., WO 92/08796, published May 29, 1992; and Lupton, S., WO 94/28143, published Dec. 8, 1994). Such marker genes can provide an added measure of control that can be advantageous in gene therapy contexts. A large variety of such vectors are known in the art and are generally available.

Vectors for use herein include viral vectors, lipid based vectors and other non-viral vectors that are capable of delivering a nucleotide encoding the therapeutic peptides described herein to the target cells. The vector can be a targeted vector, especially a targeted vector that preferentially binds to neurons and. Viral vectors for use in the application can include those that exhibit low toxicity to a target cell and induce production of therapeutically useful quantities of the therapeutic peptide in a cell specific manner.

Examples of viral vectors are those derived from adenovirus (Ad) or adeno-associated virus (AAV). Both human and non-human viral vectors can be used and the recombinant viral vector can be replication-defective in humans. Where the vector is an adenovirus, the vector can comprise a polynucleotide having a promoter operably linked to a gene encoding the therapeutic peptides and is replication-defective in humans.

Other viral vectors that can be used herein include herpes simplex virus (HSV)-based vectors. HSV vectors deleted of one or more immediate early genes (IE) are advantageous because they are generally non-cytotoxic, persist in a state similar to latency in the target cell, and afford efficient target cell transduction. Recombinant HSV vectors can incorporate approximately 30 kb of heterologous nucleic acid.

Retroviruses, such as C-type retroviruses and lentiviruses, might also be used in the application. For example, retroviral vectors may be based on murine leukemia virus (MLV). See, e.g., Hu and Pathak, Pharmacol. Rev. 52:493-511, 2000 and Fong et al., Crit. Rev. Ther. Drug Carrier Syst. 17:1-60, 2000. MLV-based vectors may contain up to 8 kb of heterologous (therapeutic) DNA in place of the viral genes. The heterologous DNA may include a tissue-specific promoter and a nucleic acid encoding the therapeutic peptide. In methods of delivery to neural cells, it may also encode a ligand to a tissue specific receptor.

Additional retroviral vectors that might be used are replication-defective lentivirus-based vectors, including human immunodeficiency (HIV)-based vectors. See, e.g., Vigna and Naldini, J. Gene Med. 5:308-316, 2000 and Miyoshi et al., J. Virol. 72:8150-8157, 1998. Lentiviral vectors are advantageous in that they are capable of infecting both actively dividing and non-dividing cells.

Lentiviral vectors for use in the application may be derived from human and non-human (including SIV) lentiviruses. Examples of lentiviral vectors include nucleic acid sequences required for vector propagation as well as a tissue-specific promoter operably linked to a therapeutic peptide encoding nucleic acid. These former may include the viral LTRs, a primer binding site, a polypurine tract, att sites, and an encapsidation site.

In some aspects, a lentiviral vector can be employed. Lentiviruses have proven capable of transducing different types of CNS neurons (Azzouz et al., (2002) J Neurosci. 22: 10302-12) and may be used in some embodiments because of their large cloning capacity.

A lentiviral vector may be packaged into any lentiviral capsid. The substitution of one particle protein with another from a different virus is referred to as “pseudotyping”. The vector capsid may contain viral envelope proteins from other viruses, including murine leukemia virus (MLV) or vesicular stomatitis virus (VSV). The use of the VSV G-protein yields a high vector titer and results in greater stability of the vector virus particles.

Alphavirus-based vectors, such as those made from semliki forest virus (SFV) and sindbis virus (SIN) might also be used in the application. Use of alphaviruses is described in Lundstrom, K., Intervirology 43:247-257, 2000 and Perri et al., Journal of Virology 74:9802-9807, 2000.

Recombinant, replication-defective alphavirus vectors are advantageous because they are capable of high-level heterologous (therapeutic) gene expression, and can infect a wide target cell range. Alphavirus replicons may be targeted to specific cell types by displaying on their virion surface a functional heterologous ligand or binding domain that would allow selective binding to target cells expressing a cognate binding partner. Alphavirus replicons may establish latency, and therefore long-term heterologous nucleic acid expression in a target cell. The replicons may also exhibit transient heterologous nucleic acid expression in the target cell.

In many of the viral vectors compatible with methods of the application, more than one promoter can be included in the vector to allow more than one heterologous gene to be expressed by the vector. Further, the vector can comprise a sequence, which encodes a signal peptide or other moiety, which facilitates expression of the therapeutic peptide from the target cell.

Other nucleotide sequence elements, which facilitate expression of the polypeptide mimetics of the acetylated αA-crystallin, acetylated αB-crystallin, and acetylated polypeptide fragments thereof and cloning of the vector are further contemplated. For example, the presence of enhancers upstream of the promoter or terminators downstream of the coding region, for example, can facilitate expression.

In accordance with another embodiment, a tissue-specific promoter can be fused to nucleotides encoding the polypeptide mimetics of the acetylated αA-crystallin, acetylated αB-crystallin, and acetylated polypeptide fragments thereof described herein. By fusing such tissue specific promoter within the adenoviral construct, transgene expression is limited to a particular tissue. The efficacy of gene expression and degree of specificity provided by tissue specific promoters can be determined, using the recombinant adenoviral system of the present application. Neuron specific promoters, such as the platelet-derived growth factor β-chain (PDGF-β) promoter and vectors, are well known in the art.

In addition to viral vector-based methods, non-viral methods may also be used to introduce a nucleic acid encoding a therapeutic peptide into a target cell. A review of non-viral methods of gene delivery is provided in Nishikawa and Huang, Human Gene Ther. 12:861-870, 2001. An example of a non-viral gene delivery method according to the application employs plasmid DNA to introduce a nucleic acid encoding a therapeutic peptide into a cell. Plasmid-based gene delivery methods are generally known in the art.

Synthetic gene transfer molecules can be designed to form multimolecular aggregates with plasmid DNA. These aggregates can be designed to bind to a target cell. Cationic amphiphiles, including lipopolyamines and cationic lipids, may be used to provide receptor-independent nucleic acid transfer into target cells.

In addition, preformed cationic liposomes or cationic lipids may be mixed with plasmid DNA to generate cell-transfecting complexes. Methods involving cationic lipid formulations are reviewed in Felgner et al., Ann. N.Y. Acad. Sci. 772:126-139, 1995 and Lasic and Templeton, Adv. Drug Delivery Rev. 20:221-266, 1996. For gene delivery, DNA may also be coupled to an amphipathic cationic peptide (Fominaya et al., J. Gene Med. 2:455-464, 2000).

Vectors that encode the expression of the therapeutic peptides can be delivered in vivo to the target cell in the form of an injectable preparation containing pharmaceutically acceptable carrier, such as saline, as necessary. Other pharmaceutical carriers, formulations and dosages can also be used in accordance with the present application.

The vector can be delivered by direct injection at an amount sufficient for the therapeutic polypeptide to be expressed to a degree, which allows for highly effective therapy. By injecting the vector directly into or about the periphery of the cell being treated, it is possible to target the vector transfection rather effectively, and to minimize loss of the recombinant vectors. This type of injection enables local transfection of a desired number of cells, especially at a site of injury, thereby maximizing therapeutic efficacy of gene transfer, and minimizing the possibility of an inflammatory response to viral proteins. Other methods of administering the vector to the target cells can be used and will depend on the specific vector employed.

The therapeutic polypeptide can be expressed for any suitable length of time within the target cell, including transient expression and stable, long-term expression. In one aspect of the application, the nucleic acid encoding the therapeutic peptide will be expressed in therapeutic amounts for a defined length of time effective to induce activity and growth of the transfected cells. In another aspect of the application, the nucleic acid encoding the therapeutic peptide will be expressed in therapeutic amounts for a defined length of time effective to restore lost function in a targeted cell after injury.

A therapeutic amount is an amount, which is capable of producing a medically desirable result in a treated animal or human. As is well known in the medical arts, dosage for any one animal or human depends on many factors, including the subject's size, body surface area, age, the particular composition to be administered, sex, time and route of administration, general health, and other drugs being administered concurrently. Specific dosages of proteins and nucleic acids can be determined readily determined by one skilled in the art using the experimental methods described below.

Vectors encoding the therapeutic peptides can often be administered less frequently than other types of therapeutics. For example, an effective amount of such a vector can range from about 0.01 mg/kg to about 5 or 10 mg/kg, inclusive; administered daily, weekly, biweekly, monthly or less frequently.

In some embodiments, compositions comprising the therapeutic polypeptides described herein can be used to treat diseases, disorders, or condition associated with pathogenic apoptosis and/or pathogenic protein aggregation.

Apoptotic diseases and related disorders, which can be treated by the therapeutic polypeptide, can include stroke, heart attack, ischemia, degenerative diseases (neuron and muscle, e.g., Alzheimer disease, Parkinson's disease, cardiomyocyte degeneration, etc), macular degeneration, hypoxia induced apoptosis, ischemia reperfusion injury, atrophy, infection by parasitic organisms (virus, bacteria, yeast, or protozoa, etc), side effects of other drugs (e.g., anti-cancer drugs), UV/X-ray irradiation, and several other pathological conditions triggering cell death signals.

In other embodiments, compositions comprising the therapeutic polypeptides can be used to treat inflammatory conditions. The inflammatory conditions for which the compositions and methods can be used are, but not limited to, surgical trauma; dry eye; allergic conjunctivitis; viral conjunctivitis; bacterial conjunctivitis; blepharitis; anterior uveitis; injury from a chemical; radiation or thermal burn; injury from penetration of a foreign body, pain in or around the eye, redness especially accompanied by pain in the eye; light sensitivity; seeing halos (colored circles or halos around lights); bulging (protrusion) of the eye; swelling of eye tissues; discharge, crusting or excessive tearing; eyelids stuck together, blood inside the front of the eye (on the colored part) or white of the eye; cataracts; pain and inflammation associated with wearing contact lenses; corneal-associated condition; conjunctival tumor excision; conjunctivitis known as Pink Eye; cornea edema after cataract surgery; corneal clouding; corneal transplantation; corneal ulcer; dry eye syndrome; dystrophies; condition associated with excimer laser phototherapeutic keratectomy; herpes simplex keratitis; keratoconus; pterygium; recurrent erosion syndrome; eye movement disorder; glaucoma; ocular oncology; oculoplastic condition resulted from cosmetic surgery, enucleation, eyelid and orbit injuries, ectropion, entropion, graves' disease, involuntary eyelid blinking; condition associated with refractive surgery; and retinal condition.

The retinal conditions for which the compositions and methods of the invention can be used are, but not limited to, macular degeneration, AIDS-related ocular disease, CMV retinitis, birdshot retinochoroidopathy (BR), choroidal melanoma, coats disease, cotton wool spots, diabetic retinopathy diabetic macular edema, cystoid macular edema, lattice degeneration, macular disease, macular degeneration, hereditary macular dystrophy, macular edema, macular hole, macular pucker, central serous chorioretinopathy, ocular histoplasmosis syndrome (OHS), posterior vitreous detachment, retinal detachment, retinal artery obstruction, retinal vein occlusion, retinoblastoma, retinopathy of prematurity (ROP), retinitis pigmentosa, retinoschisis (acquired and x-linked), Stargardt's disease, toxoplasmosis of retina or uveitis.

In still other embodiments, compositions comprising the therapeutic polypeptides can be used to treat neurological disorders. Neurological disorders may be caused by an injury to a neuron, such as a mechanical injury or an injury due to a toxic compound, by the abnormal growth or development of a neuron, or by the misregulation, such as downregulation, of an activity of a neuron. Examples of neurological disorders include traumatic or toxic injuries to peripheral or cranial nerves, spinal cord or to the brain, cranial nerves, traumatic brain injury, stroke, cerebral aneurism, and spinal cord injury. Other neurological disorders include cognitive and neurodegenerative disorders such as Alzheimer's disease, dementias related to Alzheimer's disease (such as Pick's disease), Parkinson's and other Lewy diffuse body diseases, senile dementia, Huntington's disease, Gilles de la Tourette's syndrome, multiple sclerosis, amyotrophic lateral sclerosis, hereditary motor and sensory neuropathy (Charcot-Marie-Tooth disease), diabetic neuropathy, progressive supranuclear palsy, epilepsy, and Jakob-Creutzfieldt disease. Autonomic function disorders include hypertension and sleep disorders.

The compositions described herein can also be used in a method of preserving tissues and organs for transfusions or transplantation. The cells, tissue, or organ can be stored in and/or contacted with a composition including an effective amount of therapeutic polypeptide. The effective amount of therapeutic polypeptide is an amount effective to mitigate apoptosis of the cells, tissue, or organ of interest. In some embodiments, a composition for storing cells or organs can include an effective amount of therapeutic polypeptide and an organ preservation solution.

Typically, the tissue or organ has been separated from its usual nutrient sources, e.g., the blood circulation of a living animal or person. Organ preservation solutions depend on contacting, storing and/or perfusing the organ with a supportive preservation solution designed to provide pH buffering, osmotic balance and/or some minimal nutritional support, e.g., in the form of glucose and a limited set of other basic nutrients. This approach is typically combined with reduction in organ temperature to just above the freezing point of water. This is intended to reduce the metabolic rate of organ tissues, thus slowing the consumption of nutrients and the production of waste products. Thus, in some embodiments, the therapeutic polypeptide containing compositions can be employed at the hypothermic ranges commonly used in the art, which can range from below 20° C. to about 4° C. These art-known preservative solutions include, for example, isotonic saline solutions, that may contain, in various proportions, salts, sugars, osmotic agents, local anesthetic, buffers, and other such agents, as described, simply by way of example, by Berdyaev et al., U.S. Pat. No. 5,432,053; Belzer et al., described by U.S. Pat. Nos. 4,798,824, 4,879,283; and 4,873,230; Taylor, U.S. Pat. No. 5,405,742; Dohi et al., U.S. Pat. No. 5,565,317; Stern et al., U.S. Pat. Nos. 5,370,989 and 5,552,267.

The term, “organ” as used herein encompasses both solid organs, e.g., kidney, heart, liver, lung, pancreas, as well as functional parts of organs, e.g., segments of skin, sections of artery, transplantable lobes of a liver, kidney, lung, and other organs. The term, “tissue” refers herein to viable cellular materials in an aggregate form, e.g., small portions of an organ, as well as dispersed cells, e.g., cells dispersed, isolated and/or grown from heart muscle, liver or kidney, including bone marrow cells and progeny cells, blood born stem cells and progeny, and the various other art-known blood elements, unless otherwise specified.

The invention also contemplates using a therapeutic polypeptide containing composition for localized or systemic circulatory or perfusion support for organs or tissues acutely deprived of normal blood circulation caused by trauma, e.g., infusions or temporary circulation of the inventive compositions to support a partially severed limb, or analogous conditions, until surgical repair of damaged vasculature is achieved.

The following examples are included to demonstrate preferred embodiments of the invention. It should be appreciated by those of skill in the art that the techniques disclosed in the examples, which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Previous studies have found short peptides within αA- and αB-crystallin that function as molecular chaperones, similar to the parent molecules. These peptides are ₇₀KFVIFLDVKHFSPEDLTVK₈₈ (SEQ ID NO: 13) in αA crystallin and ₇₃DRFSVNLDVKHFSPEELKVK₉₂ (SEQ ID NO: 14) in αB-crystallin. In this Example, we determined the impact of lysine acetylation on the function of these peptides. We show that the α-crystallin mini-chaperones and acetyl derivatives can inhibit apoptosis in mammalian cells by blocking cytochrome-C release from mitochondria and preventing procaspase-3 activation. Using rats, we also show that that these peptides can inhibit protein aggregation and epithelial cell apoptosis in cataracts.

### Experimental Procedures

Insulin, Hoechst, citrate synthase, sodium selenite, and a protease inhibitor cocktail were obtained from Sigma-Aldrich Chemical Co. (St. Louis, Mo.). The remaining chemicals were of analytical grade.

αA-crystallin peptides [native and acetyl: ₆₉DKFVIFLDVKHFSPEDLTVK₈₈ SEQ ID NO: 13) and ₆₉DK (acetyl) FVIFLDVKHFSPEDLTVK₈₈] (SEQ ID NO: 3) and αB-crystallin peptides [native and acetyl: ₇₃DRFSVNLDVKHFSPEELKVKV₉₃ (SEQ ID NO: 14) and ₇₃DRFSVNLDVKHFSPEELKVK(acetyl)V₉₃] (SEQ ID NO: 4) as well as the scrambled peptide DFVIDSPFKLVDLEKVHFTK (SEQ ID NO: 15) were synthesized, processed to 95-99% purity, and verified via mass spectrometry (for molecular weight and purity) by Peptide 2.0 (Chantilly, Va.).

### Chaperone Assays

Chaperone assays were performed as previously described. The ratios of the peptides: client proteins (w/w) were as follows: citrate synthase, 1:2.5; insulin, 1:10; βL-crystallin, 1:1.5; and γ-crystallin, 1:1. The scrambled αA-crystallin peptide was tested in certain assays at peptide: client protein ratios as follows: citrate synthase, 1:1.25, insulin, 1:5 to determine peptide specificity.

### Measurement of Peptide Surface Hydrophobicity

The surface hydrophobicity of the peptides was measured using 6-(ptoluidinyl) naphthalene-2-sulfonic acid (TNS) (emission: 350-520 nm, excitation: 320 nm).

### Measurement of Apoptosis

Human lens epithelial cells (HLE) were isolated from the lens of a 44-year-old donor and cultured in DMEM supplemented with 10% FBS as previously described for mouse lens epithelial cells. Cells between passages 5 and 6 were used for the experiments. Chinese hamster ovary (CHO) cells were cultured in Ham's F12 medium. The α-crystallin peptides were mixed with a cationic lipid, BIOPORTER (BP), according to the manufacturer's instructions (Polyplus transfection reagent, Illkirch, France). When the cultures reached 70-80% confluence, the cells were treated with BP either alone or with the peptides in serumfree medium and incubated for 4 h at 37° C. in a humidified 5% CO₂ atmosphere. The cells were then washed with PBS and incubated at 43° C. for 1 h (thermal stress), followed by incubation at 37° C. for 16 h for recovery. The percentage of apoptotic cells was determined by staining the cells with Hoechst. Cells incubated at 37° C. without thermal stress were used as controls.

### Quantitation of Cytoplasmic Cytochrome-C

Cell lysates were prepared in cell lysis buffer (Cell Signaling Technologies, Danvers, Mass.) with a protease inhibitor cocktail (1:100 dilution). Western blotting was performed using 10 μg of protein with a monoclonal antibody for cytochrome-C (1:1,000 dilution, Enzo Life Sciences, Farmingdale, N.Y.). The membrane was reprobed using an antibody for GAPDH (1:1,000 dilution, Millipore, Billerica, Mass.) as a loading control.

### Quantitation of Caspase-3 and Caspase-9 Activity

Cells were lysed in cell lysis buffer (Cell signaling), and lenses were homogenized in 50 mM Tris-buffered saline. Both buffers contained a protease inhibitor cocktail (1:100 dilution). An equal volume of a fluorogenic substrate solution[2× reaction buffer: 10 mM DTT and 50 μM Ac-DEVD-AFC (for caspase-3) or Ac-LEHDAFC (for caspase-9)] was added to each lysate. The lysates were then incubated for 2 h at 37° C. in the dark. The samples were analyzed with a Spectramax 4 spectrofluorometer (HORIBA Scientific, Edison, N.J.) at excitation/emission wavelengths 400/505 nm.

### Procaspase-3 Activation Assay

CHO cells were lysed with lysis buffer (Cell Signaling) containing a protease inhibitor cocktail, as previously described. The extracts were centrifuged for 10 min at 14,000 rpm, boiled for 5 min at 100° C., and analyzed by SDS-PAGE and immunoblotting using anti-β-tubulin antibody (Santa Cruz, Calif.) or antibodies for inactive caspase-3 (9665S, clone 8G10) and active-caspase-3 (9661S, clone Asp175). These antibodies were purchased from Cell Signaling.

Lens Organ Culture with Calcimycin

The animal experiments conformed to the ARVO Statement for the Use of Animals in Ophthalmic and Vision Research and were approved by the CWRU's Animal Care and Use Committee. Lenses were dissected from the eyes of ˜3-month-old C57B1/6 mice and maintained in artificial aqueous humor (AAH) (113 mM NaCl, 4.5 mM KCl, 1 mM MgCl₂, 1.5 mM CaCl₂, 6 mM D-glucose, 10 mM HEPES, 20 mM NaHCO₃, and 1:1,000 penicillin/streptomycin, pH 7.3). Lenses that developed opacification within 24 h were discarded. Native and acetyl αA- and αB crystallin peptides were added to the culture medium at a concentration of 50 μg/3.0 ml, followed by incubation of the lenses for 16 h. The medium was replaced with fresh medium containing 5 μM calcimycin, and the lenses were incubated for an additional 6 h. After this incubation period, the lenses were thoroughly washed with PBS, fixed in 1% buffered formalin overnight, and embedded in paraffin. Microtome sections (5 μm) were subsequently rehydrated and subjected to antigen retrieval via microwave irradiation in 10 mM citrate buffer (pH 6.0). Apoptosis was measured using the In situ Cell Death Detection Kit (Roche, Indianapolis, Ind.) according to the manufacturer's instructions. The sections were counterstained with DAPI to visualize nuclei.

### Selenite-Induced Cataract and Apoptosis in Lens Epithelial Cells

Neonatal Sprague-Dawley rat pups (12 days old) were used for these experiments. The pups in the experimental groups received a single subcutaneous injection of sodium selenite (4 mg/Kg body weight) on day 12. Peptides diluted from a stock in sterile water (dissolved by adding 2 μl of 10 N NaOH to a 1.0 ml stock) were injected intraperitoneally (at 2.5, 5, or 10 μg/animal) 6 h prior to selenite injection (single injection). When multiple injections were performed, one pre-selenite injection was administered, as above, followed by four additional injections on four consecutive days after the sodium selenite injection. One group served as the control (no treatment) and another as the selenite control (no peptide injection). The lenses were examined on day 17 (when the pups had opened their eyes) using a slit lamp microscope (after dilating the pupil through topical application of tropicamide) and photographed. For hematoxylin and eosin (H & E) and TUNEL staining, eyes were fixed immediately after enucleation in a 10% buffered formalin solution overnight.

### Quantitation of Water-Soluble Proteins and βB1-Crystallin

A subset of the lenses obtained from the above treatment groups was homogenized in ice-cold PBS containing 2 μM Tris(2-carboxyethyl)phosphine hydrochloride (TCEP) and 5 of a protease inhibitor cocktail in a glass homogenizer (200 μl for each lens). The homogenate was centrifuged at 20,000×g for 30 min at 4° C., and the supernatant was collected. The pellet was then homogenized again with the same buffer (100 μl, centrifuged, and the supernatant collected. The two supernatant fractions were pooled, which was considered the water-soluble fraction. The pellet was considered the water-insoluble fraction. The latter fraction was solubilized in buffer with 6 M urea. The protein concentration was measured using the BCA Protein Assay Kit (Thermo Scientific, Rockford, Ill.), with BSA as the standard. SDS-PAGE was performed using 12% reducing gels to analyze the two fractions.

βB1-Crystallin in the water-insoluble fraction (10 μg protein) was detected via western blotting using a rabbit polyclonal antibody for βB1-crystallin from Santa Cruz Biotechnology, Santa Cruz, Calif. (1:400 dilution) and a Chemiluminescence Detection Kit (Thermo Scientific).

### Markers of Oxidative Stress in Rat Lenses

The concentration of GSH in the lens was determined as previously described (29), as was the SOD1 activity in the lens homogenates. Briefly, the assay mixture (total volume=2.0 ml) contained sodium pyrophosphate buffer (52 mM, pH 8.3), 186 μM phenazine methosulfate, 300 μM nitroblue tetrazolium, and the homogenate (200 μg). The reaction was initiated by adding 5.2 mM NADH (300 μl) at a final concentration of 780 μM, and the mixture was incubated at 30° C. for 1 min. The reaction was quenched by adding 1.0 ml 5 of acetic acid, and the mixture was stirred vigorously. n-Butanol (5.0 ml) was next added to the mixture, followed by mixing and incubation at room temperature for 10 min. The mixture was finally centrifuged at 1,000×g for 10 min, after which the butanol layer was separated, and the absorbance was measured at 560 nm against a butanol blank.

### Multi-Angle Light Scattering/Dynamic Light Scattering (MALS-DLS) Experiment

Soluble lens proteins (190 μg) were prepared as described above and injected into an HPLC/TSKPWxL5000 column with PBS as the eluent at 0.75 ml/min. The remaining details regarding MALS-DLS were reported previously. The data were analyzed using ASTRA software, developed by Wyatt Technologies (Santa Barbara, Calif.).

Detection of Intraperitoneally Injected αB crystallin Peptide in Rat Lenses

Ten-day-old Sprague-Dawley rat pups (n=6) were intraperitoneally injected with 100 μg of the acetyl αB-crystallin peptide in 100 μl of sterile water on four consecutive days. The control animals (n=6) were only injected with sterile water. The animals were sacrificed 3 h after the last injection, and their lenses were removed and homogenized in 500 μl of PBS with 6 M urea. The homogenate was filtered through a 10 kDa cut-off centrifugal filter, and the filtrate was analyzed by LC-MS/MS using an Orbitrap Elite Hybrid Mass Spectrometer (Thermo Electron, San Jose, Calif.) equipped with the Waters nanoAcquity UPLC system (Waters, Taunton, Mass.). A full scan at 120,000 resolution was obtained in the Oribtrap spectrometer for the eluted peptides in the 300-1,800 amu range, followed by 20 MS/MS scans; 17 of these were used to sequence the 17 most abundant precursor ions determined from the full scan, which were fragmented in the ion trap. The remaining 3 scan events employed an inclusion list to target the acetyl αB crystallin peptide ₇₃DRFSVNLDVKHFSPEELKVK(acetyl)V₉₃: [632.84(4+), 843.45(3+), and 1264.68(2+)] (SEQ ID NO: 4). MS/MS spectra were generated through collision-induced dissociation of the peptide ions at a 35% normalized collision energy to produce a series of b- and y-ions as major fragments using dynamic exclusion with a repeat count of 2, repeat duration of 30 s, exclusion duration of 30 s, and exclusion size list of 500. The total analysis time was 90 min. Raw LC-MS/MS data were subjected to database searches using the Mascot search engine (version 2.2.0, Matrix Science) against the human SwissProt database (20,249 sequences) with the variable modifications Met oxidation and Lys acetylation and the enzyme cleavage set to none. The mass tolerance was set at 10 ppm for precursor ions and 0.8 Da for product ions. The significance threshold was p<0.05.

### Results

### αA- and αB-Crystallin Peptides Inhibit Chemical and Thermal Protein Aggregation

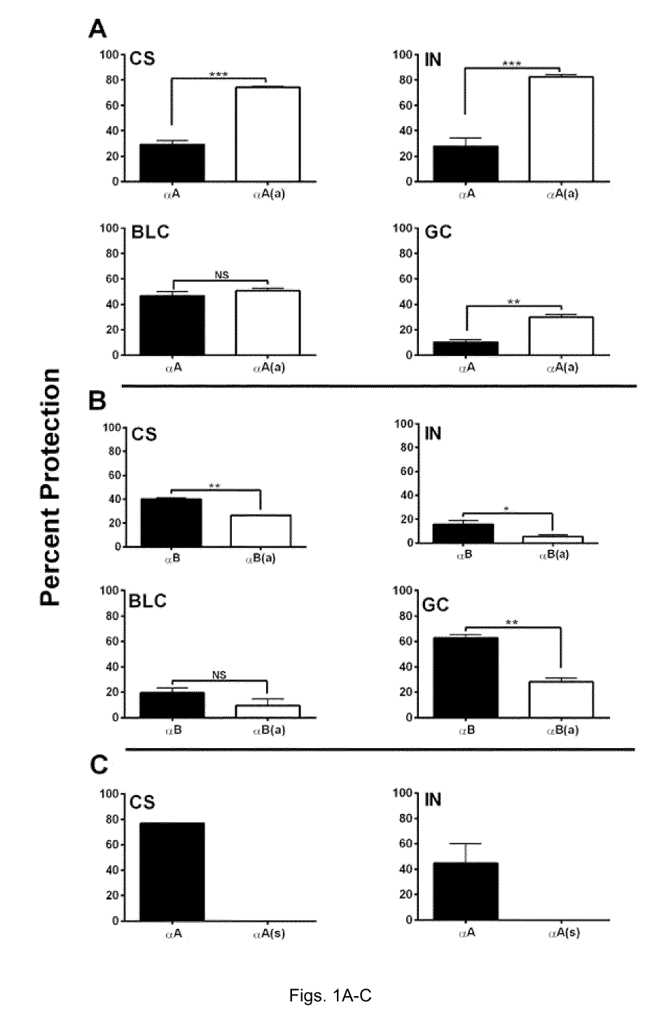

The chaperone function of the αA and αB-crystallin peptides was evaluated using four client proteins, citrate synthase (CS), insulin (IN), βL-crystallin (BLC), and γ-crystallin (GC). The chaperone function of the acetyl αA-crystallin peptide was significantly better than that of the native peptide for three of the client proteins. When βL-crystallin was used as the client protein, the results for two peptides were not different (FIG. 1A). In contrast, the acetyl αB-peptide was 10 to 30% weaker than the native peptide for the above four client proteins (FIG. 1B). However, this change in the chaperone function observed for the acetyl peptides did not correspond to surface hydrophobicity; we observed no significant difference in surface hydrophobicity between the acetyl and native peptides. To test whether the chaperone function was dependent on the specific amino acid sequence of the peptides, we used a scrambled αA-crystallin peptide in the chaperone assays. Our results showed that the scrambled peptide did not function as a chaperone for the two client proteins tested (CS and insulin), even when tested at two times higher concentration than the native and acetyl peptides (FIG. 1C). The results confirmed that the specific peptide amino acid sequence in the peptide is a rigid requirement for chaperone function.

### αA- and αB-Crystallin Peptides Inhibit the Mitochondrial Apoptosis Pathway in Thermally Stressed Cells

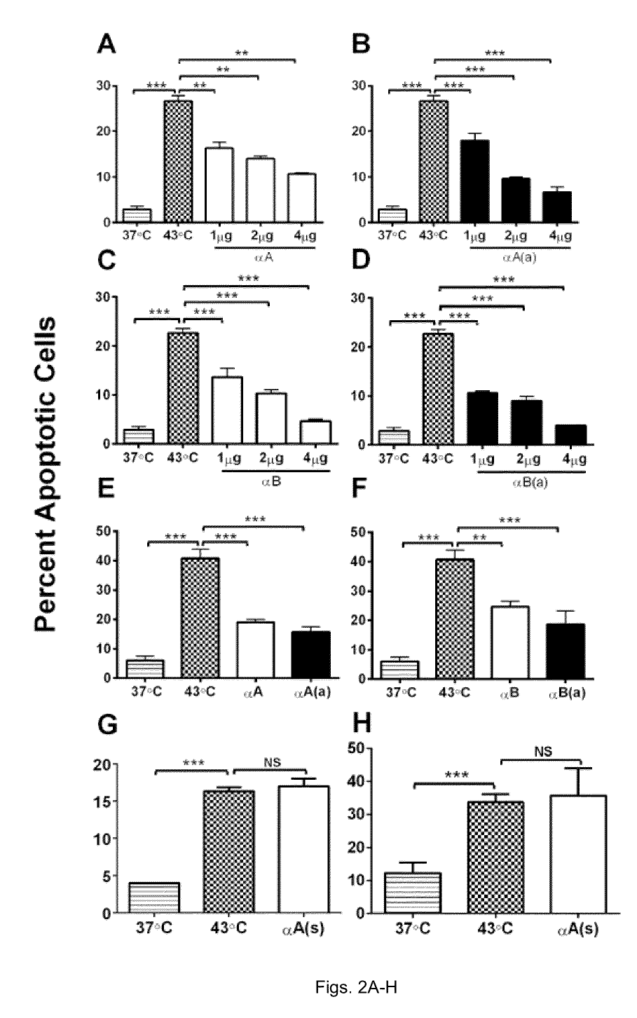

We investigated the anti-apoptotic function of the native and acetyl αA- and αB-crystallin peptides in CHO and HLE cells. The peptides were transferred to the cells using BP, and the cells were stressed at 43° C. for 1 h to induce apoptosis. The rate of hyperthermia-induced apoptosis was ˜28% in CHO cells (FIG. 2). Treatment with 1, 2, or 4 μg of the αA-crystallin peptide resulted in significant inhibition of apoptosis (by 12, 14, or 17%) (p<0.005 for each); the acetyl counterpart was at least 5% more effective than the native peptide at the 2 and 4 μg concentrations (FIGS. 2A and B). Similarly, the αB-crystallin peptide inhibited apoptosis by 8, 12, and 17% at concentrations of 1, 2, and 4 μg/ml, and the acetyl counterpart was 2-4% more effective than the native peptide at these concentrations (FIGS. 2C and D). To determine whether the anti-apoptotic function observed in CHO cells is relevant to lens epithelial cells, primary HLE cells were treated with the peptides in BP at 4 μg/ml for 4 h and held at 43° C. for 1 h. At 37° C., the cells did not undergo appreciable apoptosis. In contrast, cells at 43° C. exhibited significant apoptosis, at a rate of nearly 40% (FIG. 2E, p<0.0005). Pretreatment with the native and acetyl αA crystallin peptides reduced the rates of apoptosis to 22 and 25%, respectively (FIG. 2E), while treatment with the native and acetyl αB-crystallin peptides reduced the rates to 18 and 24%, respectively (FIG. 2F). These results indicate that the acetyl peptides are more effective than the native peptides in inhibiting apoptosis. The scrambled peptide did not inhibit apoptosis in either the CHO or HLE cells (FIGS. 2G and H).

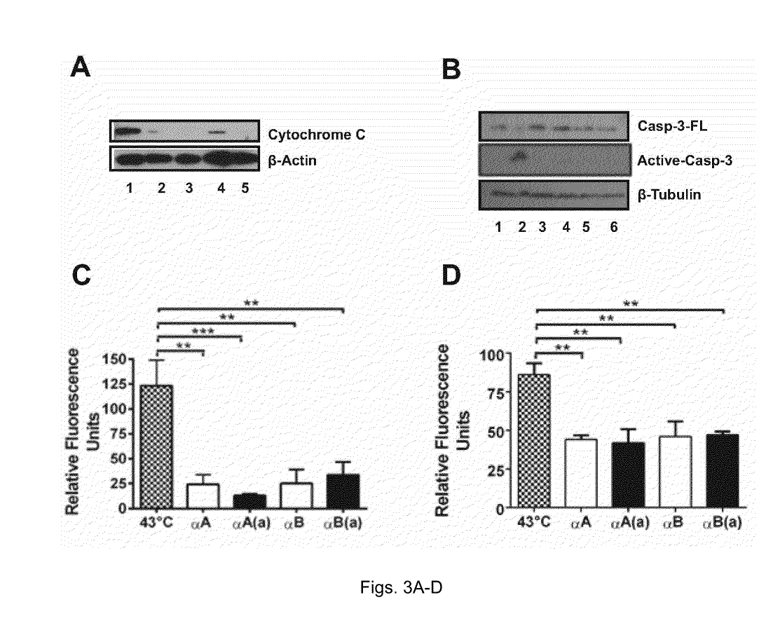

The anti-apoptotic function of the peptides primarily occurred through inhibition of the mitochondrial apoptosis pathway, which was evident from the following observations. (1) The peptides inhibited the cytosolic release of cytochrome-C from the mitochondria in thermally stressed CHO cells (FIG. 3A); the inhibition was greater for the acetyl peptides compared with the native peptides. (2) The peptides inhibited procaspase-3 maturation in thermally stressed CHO cells (FIG. 3B). Activated caspase-3 was detected only in cells that were not treated with the peptides. (3) The peptides significantly inhibited caspase-3 and -9 activity (FIGS. 3C and D). For these assays, we used peptides at a concentration of 4 μg/ml. At this concentration, the αA-peptide and its acetyl counterpart inhibited caspase-3 activity by 80 and 90% and caspase-9 activity by 51 and 53%, respectively. At similar concentrations, αB- and its acetyl counterpart inhibited caspase-3 activity by 80 and 78% and caspase-9 activity by 51 and 50%, respectively. These data show that both the αA- and αB-crystallin peptides inhibit the mitochondrial apoptosis pathway in thermally stressed cells.

### αA- and αB-Crystallin Peptides Inhibit Calcimycin-Induced Apoptosis by Inhibiting Caspases in Organ-Cultured Lenses

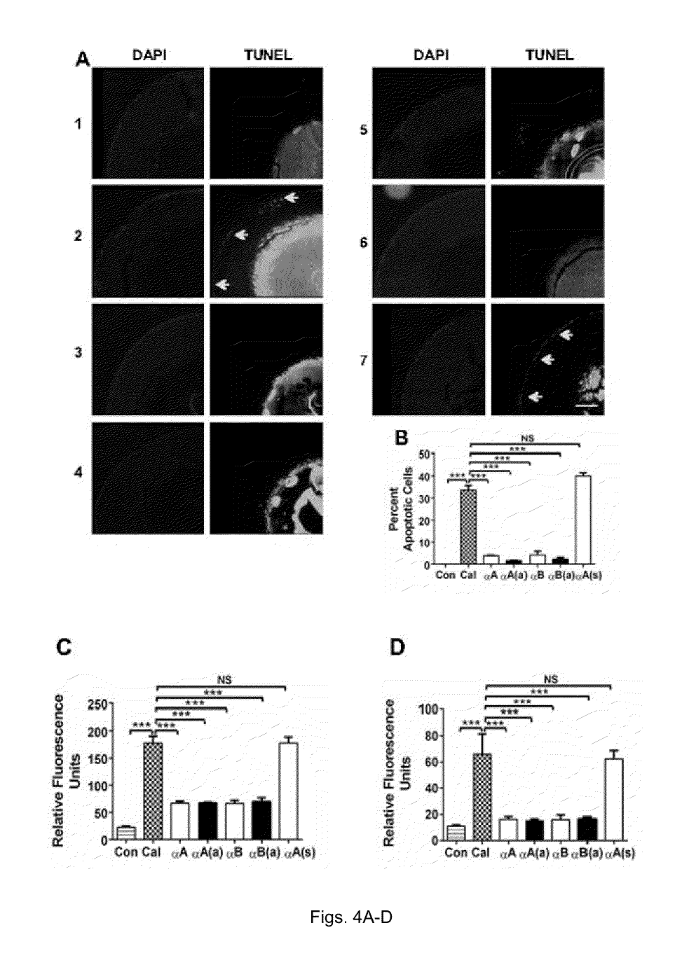

To determine whether the peptides are effective at inhibiting apoptosis in whole lenses, we used organ-cultured mouse lenses. The lenses were incubated with calcimycin to induce apoptosis, as previously reported. Incubation with calcimycin induced apoptosis of epithelial cells, as observed by TUNEL staining (FIG. 4A2). Lenses not treated with calcimycin did not show apoptosis (FIG. 4A1). Prior treatment with the peptides (native or acetyl) inhibited calcimycin-induced epithelial cell apoptosis (FIG. 4A3 to A6). Quantitation of the numbers of TUNEL-positive cells revealed that approximately 33% of lens epithelial cells were apoptotic in calcimycintreated lenses (FIG. 4B). Both the native and acetyl αA- and αB-crystallin peptides significantly inhibited apoptosis (p<0.0005), resulting in a rate of apoptosis of 3 to 5%. The scrambled peptide did not protect the cells (FIGS. 4A7 and 4B).

Apoptosis was inhibited through the mitochondrial apoptosis pathway, which was evident from the inhibition of caspase-3 and caspase-9 activity by both native and acetyl peptides (FIGS. 4C and D). These data suggest that both native and acetyl peptides can enter the lens epithelium through the capsule and inhibit mitochondrial-mediated apoptosis.

### αA- and αB-Crystallin Peptides Inhibit Selenite-Induced Cataracts in Rats

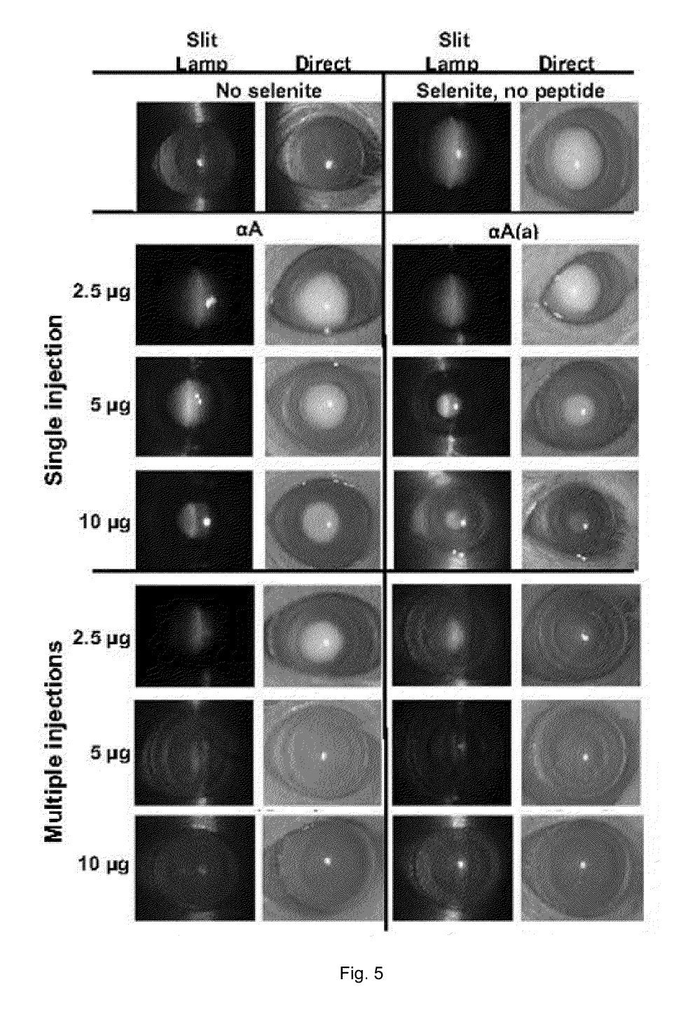

Because the peptides inhibited lens epithelial cell apoptosis in organ-cultured lenses, we next tested whether they could inhibit cataract development in an experimental animal model. Cataracts were induced by injecting sodium selenite into 12-day-old rat pups. We injected the peptides either using a single intraperitoneal injection 6 h prior to sodium selenite injection or through multiple injections. In the case of multiple injections, the first injection was given 6 h prior to sodium selenite injection, followed by subsequent injections each day for 4 days. Mature cataracts were evident when the sodium selenite-treated pups opened their eyes (FIG. 5, top right panels). When administered in a single 2.5 μg injection, both the native and acetyl αA-peptides were ineffective. At 5 and 10 μg doses, the acetyl peptide was more effective than the native peptide in inhibiting cataract development. When administered as multiple 2.5 μg injections, the acetyl peptide partially inhibited cataract development. At 5 μg, both peptides strongly inhibited cataracts, and at 10 μg, they entirely inhibited cataracts.

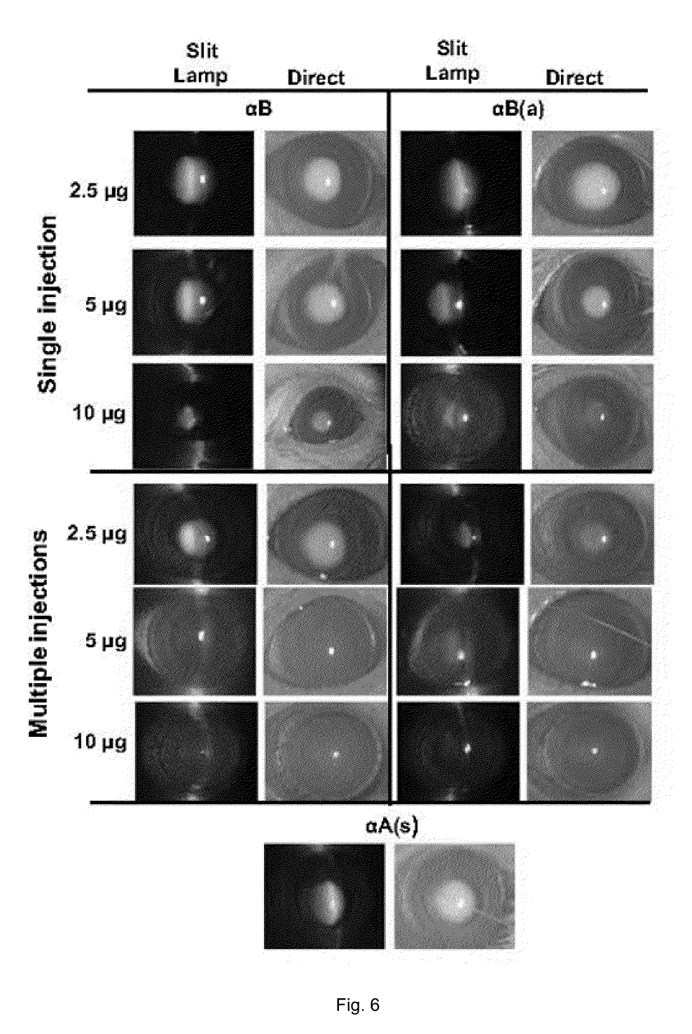

We also tested the native and acetyl αB-crystallin peptides for selenite-cataract inhibition. Under the single injection regimen, they were both ineffective at 2.5 μg, whereas mild inhibition of cataracts was observed at a dose of 5 μg of the acetyl peptide, but not with the native peptide. However, at 10 μg, both peptides strongly inhibited cataract development (FIG. 6). Under the multiple injection regimen, mild cataract inhibition was evident following treatment with 2.5 μg of the acetyl peptide, and cataracts were significantly inhibited at doses of 5 and 10 μg. To test whether the effects of the crystallin peptides were direct, we injected 10 μg of the scrambled αA crystallin peptide using the multiple injection regimen, which was observed to be entirely ineffective (FIG. 6, bottom panels), confirming that the specific amino acid sequence of the native protein was necessary for preventing cataracts.

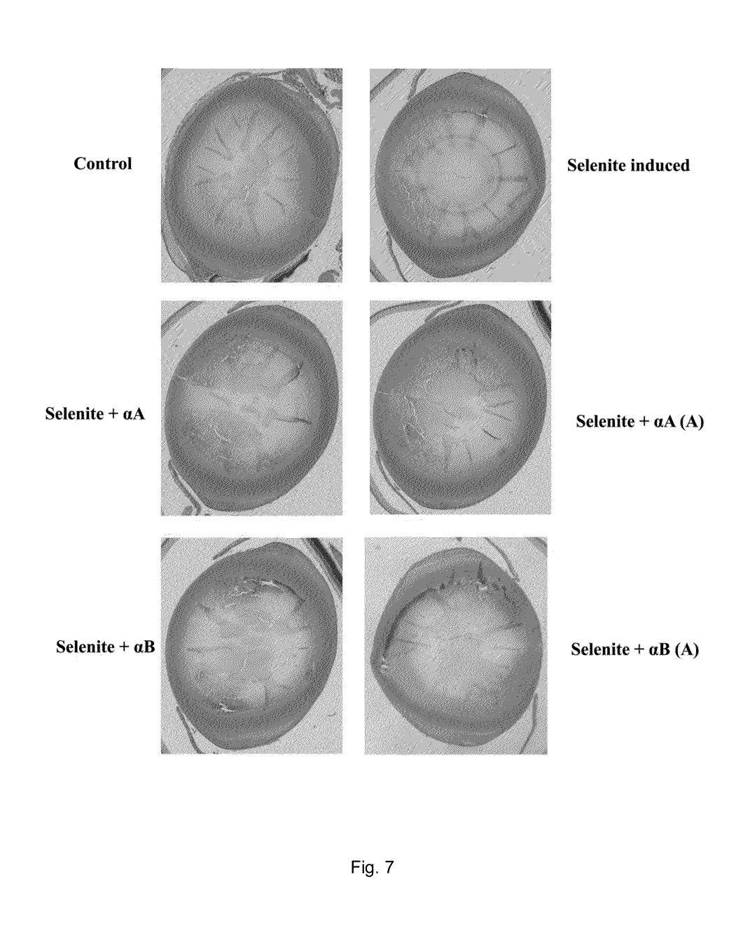

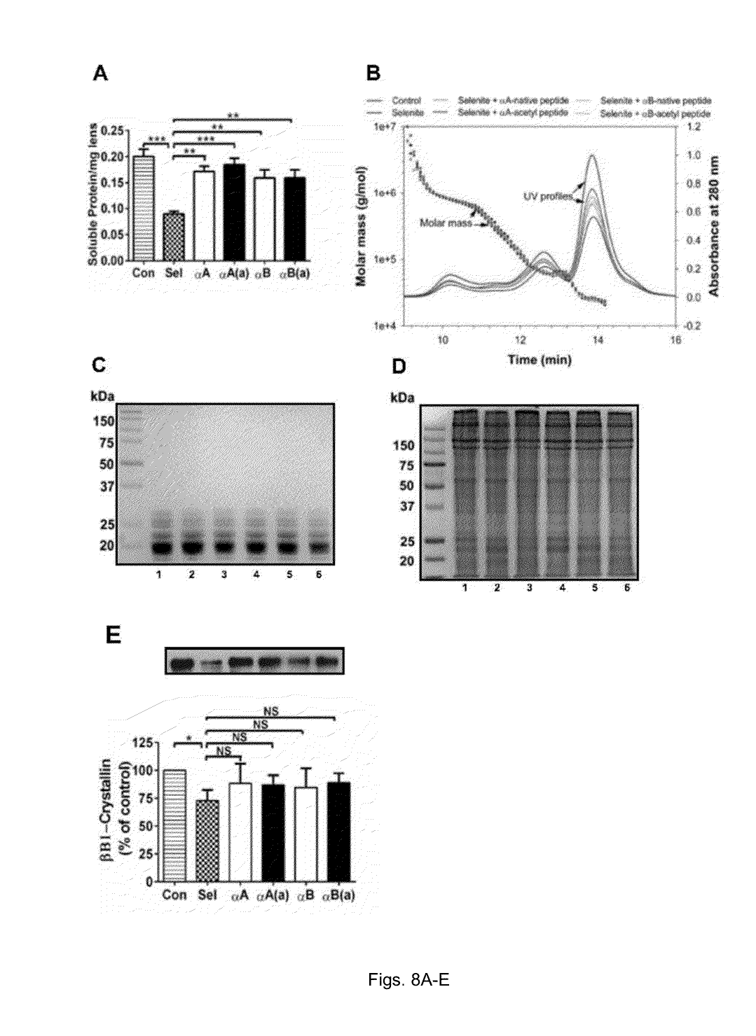

A histological examination of the selenite-treated cataract lenses showed protein aggregates in the nuclear region (FIG. 7). Peptide treatment corrected this change to control levels. Extraction of lenses in buffer, followed by protein quantity estimation, revealed a significant (p<0.0005; more than 50%) reduction of soluble protein associated with the selenite-induced cataracts (FIG. 8A). This reduction was significantly inhibited (70-80%) by both the native and acetyl peptides. Analyses of the water-soluble protein fraction via MALS-DLS revealed no covalent protein aggregation in the seleniteinduced cataracts, and peptide treatment did not have an apparent effect on the protein profile (FIG. 8B). This was further confirmed via SDS-PAGE, which showed similar protein profiles for the water-soluble and insoluble fractions (FIGS. 8C and D). In selenite-induced cataracts in rats, m-calpain or calpain II is activated, which cleaves crystallins (βB1-crystallin in particular). To determine whether the peptides inhibited such cleavage, we subjected the water-insoluble fraction to western blotting using a βB1-crystallin antibody. The results showed that βB1-crystallin was significantly reduced in selenite-induced cataracts (p<0.05), and both the acetyl and native crystallin peptides inhibited this reduction, although the effect was not significant (FIG. 8E).

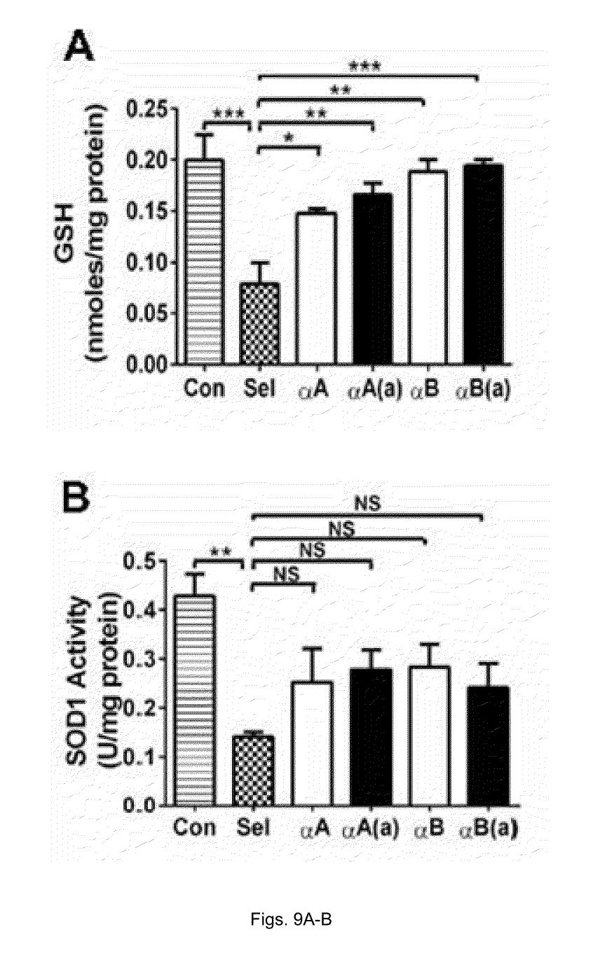

Biochemical analyses of the lenses showed that the levels of GSH and the antioxidant enzyme SOD1 were significantly reduced in selenite-induced cataracts, which was mostly corrected by peptide administration (significantly for GSH; FIGS. 9A and B). This corrective effect was slightly greater for the acetyl peptides compared with the native peptides.

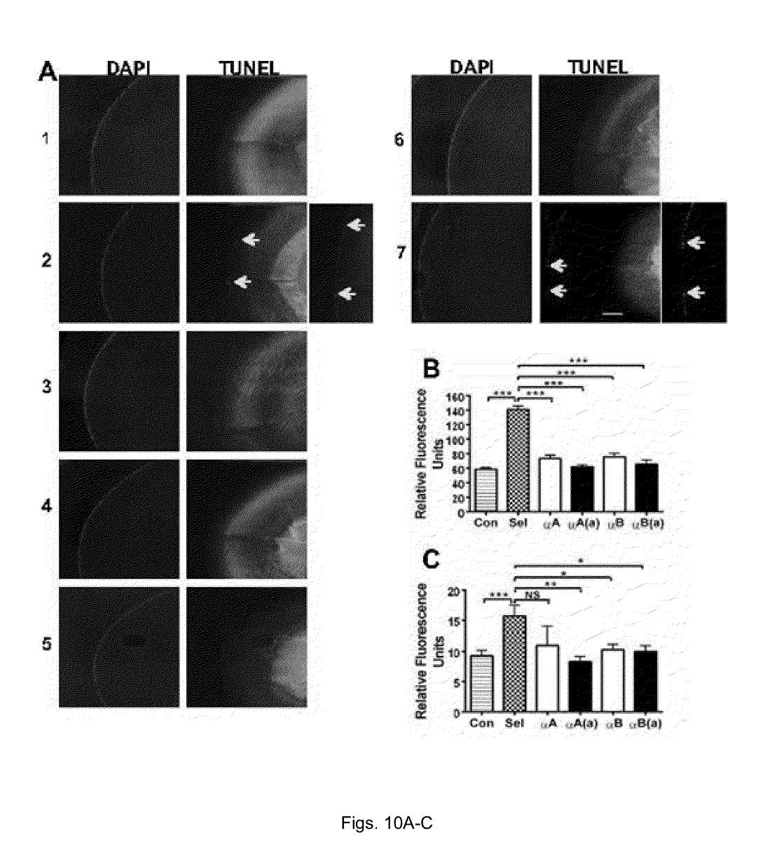

In situ TUNEL staining showed apoptosis of lens epithelial cells in selenite induced cataracts (FIG. 10A2, arrows), which was not observed in control lenses (FIG. 10A1). Both the native and acetyl αA- and αB-crystallin peptides inhibited apoptosis (FIG. 10A3-A6). The activity of caspase-3 and 9 was significantly elevated in selenite induced cataracts (p<0.0005), which was significantly reduced by the peptide treatment (except for the native αA-peptide in the case of caspase-9) (FIGS. 10B and C).

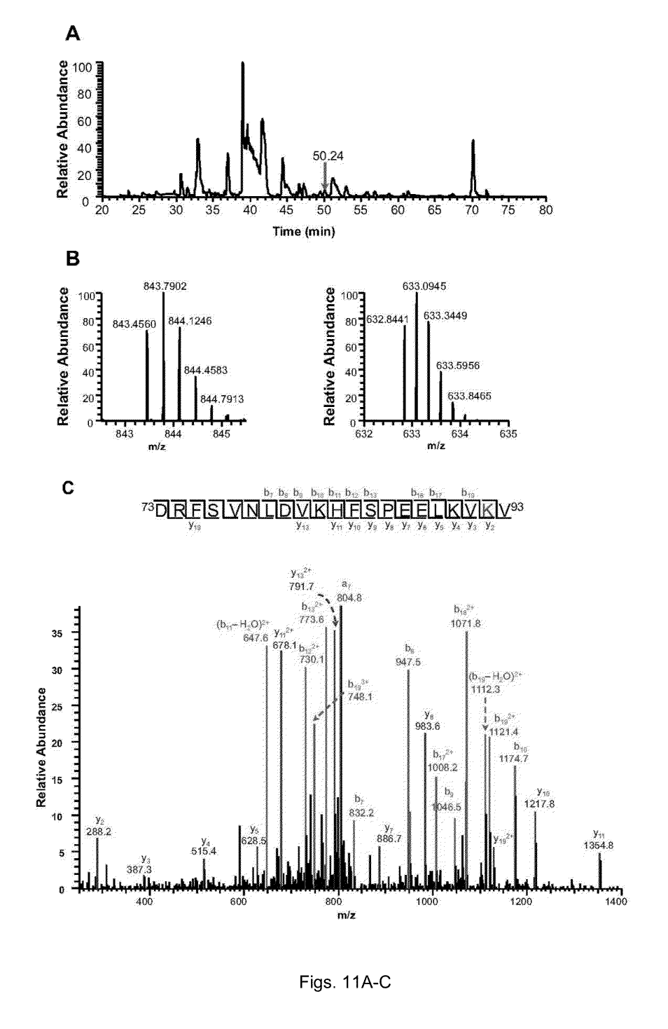

The above effects were due to the translocation of the intraperitoneally injected peptide to the lens, which was verified by mass spectrometry. The low molecular weight isolate from pooled lenses from rat pups administered four injections of 100 μg of the acetyl αB-peptide was analyzed for presence of the peptide. We detected the peptide in the lenses of peptide-injected animals (FIG. 11A). Peptides with multiple positively charged residues, such as Arg, His, and Lys, are often observed to be highly charged in ESI mass spectra. For the αB peptide, both triply and quadruply charged parent ions, 843.4560(3+) and 632.8441(4+) (FIG. 11B) were detected in the full mass spectrum (error <2 ppm). The obtained tandem mass spectra (MS/MS) confirmed the peptide sequence. As shown in FIG. 11C, the b series ions from b7 through b19 were all unmodified, which demonstrated that acetylation did not occur at K83 or K90, whereas the y2 ion observed at 288.2 (+42 Da) confirmed that K92 is acetylated. This peptide was not detected in the control sample, even though the m/z values were included in the parent ion mass scan.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

Having described the invention, we claim:

1. A method of inhibiting, reducing, and/or treating pathological apoptosis and/or protein aggregation in a subject, the method comprising:
administering to the subject an amount of a therapeutic polypeptide effective to inhibit, reduce, and/or treat the pathological apoptosis and/or protein aggregation, the therapeutic polypeptide including at least one of acetylated αA-crystallin, acetylated αB-crystallin, acetylated fragments thereof having molecular chaperone activity, and/or polypeptide mimetics thereof that can inhibit pathological protein aggregation and/or pathological apoptosis.

2. The method of claim 1, wherein the therapeutic polypeptide has an amino acid sequence with a sequence identity of SEQ ID NO: 9, SEQ ID NO: 10, SEQ ID NO: 11 or SEQ ID NO: 12.

3. The method of claim 1 wherein the therapeutic polypeptide has an amino acid sequence with a sequence identity selected from the group consisting of SEQ ID NO: 1, SEQ ID NO: 2, SEQ ID NO: 3 and SEQ ID NO: 4.

4. The method of claim 1 wherein the therapeutic polypeptide has an amino acid sequence with a sequence identity selected from the group consisting of SEQ ID NO: 5, SEQ ID NO: 6, SEQ ID NO: 7, and SEQ ID NO: 8.

5. The method of claim 1, wherein the therapeutic polypeptide has an amino acid sequence with a sequence identity selected from the group consisting of SEQ ID NO: 3, SEQ ID NO: 4, SEQ ID NO: 7, and SEQ ID NO: 8.

6. The method of claim 1, the pathological apoptosis and/or pathological protein aggregation being associated with or resulting from an optical neuropathy, glaucoma, or cataracts.

7. The method of claim 1, the pathological apoptosis and/or pathological protein aggregation being associated with or resulting from Alzheimer's disease.

8. The method of claim 1, the pathological apoptosis and/or pathological protein aggregation being associated with or resulting from a brain injury.

9. The method of claim 1, the pathological apoptosis and/or pathological protein aggregation being associated with or resulting from an inflammatory disease.

10. The method of claim 1, the therapeutic polypeptide being administered systemically to the subject.

11. The method of claim 1, the therapeutically polypeptide being administered locally.

12. A method of inhibiting apoptosis of retinal pigment epithelial cell in a subject, the method comprising:
administering to the cell an amount of a therapeutic polypeptide effective to inhibit pathological apoptosis of the cell, the therapeutic polypeptide including at least one of acetylated αA-crystallin, acetylated αB-crystallin, acetylated fragments thereof having molecular chaperone activity, and/or polypeptide mimetics thereof.

13. The method of claim 12, wherein the therapeutic polypeptide has an amino acid sequence with a sequence identity of SEQ ID NO: 9, SEQ ID NO: 10, SEQ ID NO: 11 or SEQ ID NO: 12.

14. The method of claim 12 wherein the therapeutic polypeptide has an amino acid sequence with a sequence identity selected from the group consisting of SEQ ID NO: 1, SEQ ID NO: 2, SEQ ID NO: 3 and SEQ ID NO: 4.

15. The method of claim 12 wherein the therapeutic polypeptide has an amino acid sequence with a sequence identity selected from the group consisting of SEQ ID NO: 5, SEQ ID NO: 6, SEQ ID NO: 7, and SEQ ID NO: 8.

16. The method of claim 12, wherein the therapeutic polypeptide has an amino acid sequence with a sequence identity selected from the group consisting of SEQ ID NO: 3, SEQ ID NO: 4, SEQ ID NO: 7, and SEQ ID NO: 8.

17. The method of claim 12, the pathological apoptosis and/or pathological protein aggregation being associated with or resulting from an optical neuropathy, glaucoma, or cataracts.

18. The method of claim 12, the therapeutic polypeptide being administered systemically to the subject.

19. The method of claim 12, the therapeutically polypeptide being administered locally.

20. A method of treating a cataract in a subject, the method comprising:
administering to the subject's eye an amount of a therapeutic polypeptide effective to inhibit protein aggregation and/or epithelial cell apoptosis in the lens of the subject's eye, the therapeutic polypeptide including at least one of acetylated αA-crystallin, acetylated αB-crystallin, acetylated fragments thereof having molecular chaperone activity, and/or polypeptide mimetics thereof.

21. The method of claim 20, wherein the therapeutic polypeptide has an amino acid sequence with a sequence identity of SEQ ID NO: 9, SEQ ID NO: 10, SEQ ID NO: 11 or SEQ ID NO: 12.

22. The method of claim 20 wherein the therapeutic polypeptide has an amino acid sequence with a sequence identity selected from the group consisting of SEQ ID NO: 1, SEQ ID NO: 2, SEQ ID NO: 3 and SEQ ID NO: 4.

23. The method of claim 20 wherein the therapeutic polypeptide has an amino acid sequence with a sequence identity selected from the group consisting of SEQ ID NO: 5, SEQ ID NO: 6, SEQ ID NO: 7, and SEQ ID NO: 8.

24. The method of claim 20, wherein the therapeutic polypeptide has an amino acid sequence with a sequence identity selected from the group consisting of SEQ ID NO: 3, SEQ ID NO: 4, SEQ ID NO: 7, and SEQ ID NO: 8.


SEQUENCE LISTING (15 sequences)

SEQ ID NO: 1 - protein, 173 aa, Homo sapiens
  MOD_RES (70)..(70): X aais an acetylated lysine
MDVTIQHPWFKRTLGPFYPSRLFDQFFGEGLFEYDLLPFLSSTISPYYRQSLFRTVLDSGISEVRSDRDXFVIFLDVKHFSPEDLTVKVQDDFVEIHGKHNERQDDHGYISREFHRRYRLPSNVDQSALSCSLSADGMLTFCGPKIQTGLDATHAERAIPVSREEKPTSAPSS

SEQ ID NO: 2 - protein, 175 aa, Homo sapiens
  MOD_RES (92)..(92): Xaa is an acetylated lysine
MDIAIHHPWIRRPFFPFHSPSRLFDQFFGEHLLESDLFPTSTSLSPFYLRPPSFLRAPSWFDTGLSEMRLEKDRFSVNLDVKHFSPEELKVXVLGDVIEVHGKHEERQDEHGFISREFHRKYRIPADVDPLTITSSLSSDGVLTVNGPRKQVSGPERTIPITREEKPAVTAAPKK

SEQ ID NO: 3 - protein, 20 aa, Homo sapiens
  MOD_RES (2)..(2): Xaa is an acetylated lysine
DXFVIFLDVKHFSPEDLTVK

SEQ ID NO: 4 - protein, 21 aa, Homo sapiens
  MOD_RES (20)..(20): Xaa is an acetylated lysine
DRFSVNLDVKHFSPEELKVXV

SEQ ID NO: 5 - protein, 173 aa, Homo sapiens
MDVTIQHPWFKRTLGPFYPSRLFDQFFGEGLFEYDLLPFLSSTISPYYRQSLFRTVLDSGISEVRSDRDQFVIFLDVKHFSPEDLTVKVQDDFVEIHGKHNERQDDHGYISREFHRRYRLPSNVDQSALSCSLSADGMLTFCGPKIQTGLDATHAERAIPVSREEKPTSAPSS

SEQ ID NO: 6 - protein, 175 aa, Homo sapiens
MDIAIHHPWIRRPFFPFHSPSRLFDQFFGEHLLESDLFPTSTSLSPFYLRPPSFLRAPSWFDTGLSEMRLEKDRFSVNLDVKHFSPEELKVQVLGDVIEVHGKHEERQDEHGFISREFHRKYRIPADVDPLTITSSLSSDGVLTVNGPRKQVSGPERTIPITREEKPAVTAAPKK

SEQ ID NO: 7 - protein, 20 aa, Homo sapiens
DQFVIFLDVKHFSPEDLTVK

SEQ ID NO: 8 - protein, 21 aa, Homo sapiens
DRFSVNLDVKHFSPEELKVQV

SEQ ID NO: 9 - protein, 173 aa, Homo sapiens; identical to SEQ ID NO: 1
  MOD_RES (70)..(70): Xaa is an acetylated lysine or glutamine

SEQ ID NO: 10 - protein, 175 aa, Homo sapiens; identical to SEQ ID NO: 2
  MOD_RES (92)..(92): Xaa is an acetylated lysine or glutamine

SEQ ID NO: 11 - protein, 20 aa, Homo sapiens
  MOD_RES (2)..(2): Xaa is an acetylated lysine or glutamine
DXFVIFLDVKHFSPEDLTVK

SEQ ID NO: 12 - protein, 21 aa, Homo sapiens
  MOD_RES (20)..(20): Xaa is an acetylated lysine or glutamine
DRFSVNLDVKHFSPEELKVXV

SEQ ID NO: 13 - protein, 20 aa, Homo sapiens
DKFVIFLDVKHFSPEDLTVK

SEQ ID NO: 14 - protein, 21 aa, Homo sapiens
DRFSVNLDVKHFSPEELKVKV

SEQ ID NO: 15 - protein, 20 aa, Homo sapiens
DFVIDSPFKLVDLEKVHFTK